(19) United States
(12) Patent Application Publication
(10) Pub. No.: US 2016/0135904 A1
(43) Pub. Date: May 19, 2016

(54) SYSTEM AND METHOD FOR REAL TIME TRACKING AND MODELING OF SURGICAL SITE

(71) Applicant: Navigate Surgical Technologies, Inc., Vancouver (CA)

(72) Inventors: Ehud (Udi) DAON, North Vancouver (CA); Martin Gregory BECKETT, Bowen Island (CA)

(21) Appl. No.: 15/004,653

(22) Filed: Jan. 22, 2016

Related U.S. Application Data
(63) Continuation-in-part of application No. 14/645,927, filed on Mar. 12, 2015.
Application No. 14/645,927 is a continuation-in-part of application No. 14/599,149, filed on Jan. 16, 2015.
Application No. 14/599,149 is a division of application No. 13/571,285, filed on Aug. 9, 2012.
Application No. 13/571,285 is a division of application No. 13/571,284, filed on Aug. 9, 2012, now Pat. No. 8,938,282.
Application No. 13/571,284 is a continuation-in-part of application No. 13/822,358, filed on Mar. 12, 2013.
(60) Provisional application No. 61/952,832, filed on Mar. 13, 2014.
(60) Provisional application No. 61/553,058, filed on Oct. 28, 2011.
(60) Provisional application No. 61/671,861, filed on Jul. 16, 2012.

(51) Int. Cl. A61B 34/20; A61B 90/00

(52) U.S. Cl. CPC A61B 34/20; A61B 90/39; A61B 2034/2055; A61B 2090/3983; A61B 2090/3937

(57) ABSTRACT

The present invention involves a surgical site monitoring system and associated method of use employing passive vectorized tracking markers attached to a surgical implement, an intra-oral mapping device and/or a scan-visible passive vectorized fiducial reference fixed to a surgical site. A non-stereo optical tracker obtains image information about the tracking markers and uses either markings on or shapes of the tracking markers to determine from the image information the relative 3D locations and orientations of the surgical implement, mapping device and/or fiducial reference. A scan of the surgical site prior to a surgical procedure with the fiducial reference attached is used to obtain scan data. The system and method allow the scan data and intra-oral maps of the surgery site by the mapping device to be three-dimensionally superimposed and, in some embodiments, allows surgery to be tracked. In some embodiments, the superimposition may be done in real time during surgery.

### CROSS-REFERENCE TO RELATED APPLICATIONS

The present application is a Continuation-in-part of U.S. patent application Ser. No. 14/645,927 which claims priority under 35 U.S.C. §119(e) of U.S. Provisional Patent Application Ser. No. 61/952,832, filed Mar. 13, 2014; and is a continuation-in-part of U.S. patent application Ser. No. 14/599,149, filed Jan. 16, 2015, which is a divisional application of U.S. patent application Ser. No. 13/571,284, filed Oct. 28, 2011, and also claims priority to Ser. No. 13/822,358, filed Mar. 12, 2013, both of which claim priority under 35 U.S.C. §119(e) of U.S. Provisional Patent Application Ser. No. 61/553,056, filed Oct. 28, 2011, and 61/616,718, filed Mar. 28, 2012.

### BACKGROUND OF THE INVENTION

1. Field of the Invention

The invention relates to location monitoring hardware and software systems. More specifically, the field of the invention is that of surgical equipment and software for monitoring surgical conditions.

2. Description of the Related Art

Visual and other sensory systems are known, with such systems being capable of both observing and monitoring surgical procedures. With such observation and monitoring systems, computer aided surgeries are now possible, and in fact are being routinely performed. In such procedures, the computer software interacts with both clinical images of the patient and observed surgical images from the current surgical procedure to provide guidance to the physician in conducting the surgery. For example, in one known system a carrier assembly bears at least one fiducial marker onto an attachment element in a precisely repeatable position with respect to a patient's jaw bone, employing the carrier assembly for providing registration between the fiducial marker and the patient's jaw bone and implanting the tooth implant by employing a tracking system which uses the registration to guide a drilling assembly. With this relatively new computer implemented technology, further improvements may further advance the effectiveness of surgical procedures.

### SUMMARY OF THE INVENTION

The present invention involves embodiments of surgical hardware and software monitoring system and method which allows for surgical planning while the patient is available for surgery, for example while the patient is being prepared for surgery so that the system may model the surgical site. In one embodiment, the model may be used to track contemplated surgical procedures and warn the physician regarding possible boundary violations that would indicate an inappropriate location in a surgical procedure. In another embodiment, the hardware may track the movement of instruments during the procedure and in reference to the model to enhance observation of the procedure. In this way, physicians are provided an additional tool to improve surgical planning and performance.

The system uses a particularly configured passive vectorized fiducial reference, to orient the monitoring system with regard to the critical area. The fiducial reference is attached to a location near the intended surgical area. For example, in the example of a dental surgery, a splint may be used to securely locate the fiducial reference near the surgical area. The fiducial reference may then be used as a point of reference, or a fiducial, for the further image processing of the surgical site. The fiducial reference may be identified relative to other portions of the surgical area by having a recognizable fiducial marker apparent in the scan.

The embodiments of the invention involve automatically computing the three-dimensional location of the patient by means of a tracking device that may be a passive vectorized tracking marker. The tracking marker may be attached in fixed spatial relation either directly to the fiducial reference, or attached to the fiducial reference via a tracking pole that itself may have a distinct three-dimensional shape. In the dental surgery example, a tracking pole is mechanically connected to the base of the fiducial reference that is in turn fixed in the patient's mouth. Each tracking pole device has a particular observation pattern, located either on itself or on a suitable passive vectorized tracking marker, and a particular geometrical connection to the base, which the computer software recognizes as corresponding to a particular geometry for subsequent location calculations. Although individual tracking pole devices have distinct configurations, they may all share the same connection base and thus may be used with any passive vectorized fiducial reference. The particular tracking information calculations are dictated by the particular tracking pole used, and actual patient location is calculated accordingly. Thus, tracking pole devices may be interchanged and calculation of the location remains the same. This provides, in the case of dental surgery, automatic recognition of the patient head location in space. Alternatively, a sensor device, or a tracker, may be in a known position relative to the fiducial key and its tracking pole, so that the current data image may be mapped to the scan image items.

The vectorized fiducial reference and each tracking pole or associated passive vectorized tracking marker may have a pattern made of radio opaque material so that when imaging information is scanned by the software, the particular items are recognized. Typically, each instrument used in the procedure has a unique pattern on its associated tracking marker so that the tracker information identifies the instrument. The software creates a model of the surgical site, in one embodiment a coordinate system, according to the location and orientation of the patterns on the fiducial reference and/or tracking pole(s) or their attached tracking markers. By way of example, in the embodiment where the fiducial reference has an associated pre-assigned pattern, analysis software interpreting image information from the tracker may recognize the pattern and may select the site of the base of the fiducial to be at the location where the fiducial reference is attached to a splint. If the fiducial key does not have an associated pattern, a fiducial site is designated. In the dental example this can be at a particular spatial relation to the tooth, and a splint location can be automatically designed for placement of the fiducial reference.

An in situ imager, tagged with a suitable passive vectorized tracking marker, provides live imagery of the surgical site. The tracking marker on the imager is tracked by the tracker of the system. Since the mutual relative locations and orientations of the in situ imager and the tracking marker are known, the controller of the system may derive the location and orientation of the imager by tracking the marker on the imager. This allows the exact view of the imager to be computed and live imagery from the in situ imager to be overlaid on a model of the surgical site in real time.

In a first aspect, a position monitoring system is presented for a surgical procedure comprising: a single passive vectorized fiducial reference adapted to be fixed to a surgical site of a surgical patient; an imaging sensor adapted for disposing proximate the surgical site and adapted for obtaining live images of the surgical site; an illuminator adapted for illuminating the surgical site with radiation; a first passive vectorized tracking marker rigidly attached in a predetermined fixed three-dimensional position and orientation relative to the single fiducial reference; a second passive vectorized tracking marker rigidly attached in a predetermined fixed three-dimensional position and orientation relative to the imaging sensor; a tracker configured and disposed for obtaining image information of at least the first and second tracking markers; scan data of the surgical site before the surgical procedure with the single fiducial reference fixed to the surgical site; a controller data-wise coupled to the tracker and to the imaging sensor and comprising a processor with memory and a software program having a series of instructions which when executed by the processor determines from the image information current positions and orientations of the first and second tracking markers, and relates the scan data to the current three-dimensional position and orientation of the single fiducial reference and to the current live image of the surgical site; and a display system data-wise coupled to the controller and adapted to show during the surgical procedure the current live image of the surgical site in three-dimensional spatial relationship relative to the scan data. The tracker may be an optical tracker. More specifically, the tracker may be a non-stereo optical tracker. In other embodiments, the tracker may be a stereo optical tracker. The single passive vectorized fiducial reference may be at least partially non-visible when fixed to the surgical site.

The system may further comprise a surgical implement bearing a third passive vectorized tracking marker, wherein the tracker is further configured and disposed for obtaining image information of the third tracking marker; the software program has a further series of instructions which when executed by the processor determines from the image information the current position and orientation of the third tracking marker and relates the scan data to the current position and orientation of the surgical implement.

In another aspect, a method is presented for monitoring a surgical site, comprising: removably attaching a single passive vectorized fiducial reference to a fiducial location proximate a surgical site, the fiducial reference having at least one of a marking and a shape perceptible on a scan; creating prior to the surgical procedure a scan of the surgical site and the fiducial location with the single fiducial reference attached; removably and rigidly attaching to the single fiducial reference a first passive vectorized tracking marker disposed within a field of view of a tracker; disposing proximate the surgical site an imaging sensor bearing a second passive vectorized tracking marker disposed in the field of view of tracker; receiving from the tracker image information of at least the surgical site and the first and second tracking markers; obtaining from the imaging sensor live images of the surgical site; determining from the scan data, the image information, and the live images of the surgical site a continuously updated 3-dimensional model of the surgical site overlaid with live imagery of the surgical site. The removably attaching the single fiducial reference may be removably attaching the single fiducial reference to be disposed at least partly non-visible to the tracker. The receiving image information may be receiving optical image information. In particular, the receiving optical image information may be receiving non-stereo optical image information. The obtaining live images may comprise one of obtaining live optical images and obtaining live X-ray transmission images. The obtaining live optical images may comprise be one or both of obtaining live optical images based on reflected light and obtaining live fluoroscopic images.

The determining the continuously updated three-dimensional model of the surgical site may comprise: determining from the first scan data a three-dimensional location and orientation of the single fiducial reference relative to the surgical site based on at least one of markings on and the shape of the single fiducial reference; determining from the image information three-dimensional location and orientation information about the first and second tracking markers; and calculating from the three-dimensional locations and orientations of the first and second tracking markers the corresponding three-dimensional locations and orientations of the single fiducial reference and imaging sensor, respectively.

The determining the continuously updated three-dimensional model of the surgical site may further comprise: determining from the image information three-dimensional location and orientation information about a third passive vectorized tracking marker fixedly attached to a surgical implement; and calculating from the three-dimensional location and orientation of the third tracking marker the corresponding three-dimensional location and orientation of the surgical implement.

In a further aspect of the invention, a monitoring system is provided for a surgical site comprising: a single passive vectorized scan-visible fiducial reference adapted to be fixed proximate an oral surgical site of a surgical patient; an intra-oral mapping device adapted to be disposed intra-orally proximate the surgical site and adapted to obtain current mapping information of an intra-oral mapping area including the surgical site and the fiducial reference; pre-surgical scan data of the surgical site with the fiducial reference fixed proximate the surgical site, the scan data including the fiducial reference; a controller in data communication with the intra-oral mapping device and comprising a processor with memory and a software program comprising a series of instructions which when executed by the processor determines from the current mapping information a current three-dimensional spatial position and orientation of the fiducial reference relative to the intra-oral mapping device, and spatially relates the scan data to the current mapping information based on the current three-dimensional spatial position and orientation of the single fiducial reference; and a display system in data communication with the controller and adapted to display the current mapping information of the surgical site superimposed on the scan data.

In a first embodiment, the monitoring system may further comprise: a first passive vectorized tracking marker rigidly and removably disposed in a predetermined fixed three-dimensional spatial position and orientation relative to the single fiducial reference; a non-stereo optical tracker in data communication with the controller and configured and disposed for obtaining from a field of view of the tracker real time image information of at least the first tracking marker; and the software program comprising a further series of instructions which when executed by the processor determines from the real time image information a current three-dimensional spatial position and orientation of the first tracking marker, and relates the scan data and current intra-oral mapping information to the current three-dimensional spatial position and orientation of the first tracking marker.

In the same first embodiment, the system may further comprise a surgical implement bearing a second passive vectorized tracking marker in fixed three-dimensional spatial relationship with a working tip of the surgical implement and disposed within the field of view of the tracker, wherein the real time image information of at least the first tracking marker further includes information of the second tracking marker, and the software program comprises yet a further series of instructions which when executed by the processor determines from the real time image information current positions and orientations of the second tracking marker, and relates a position and orientation of a working tip of the surgical implement to the surgical site based on the real time information of the second tracking marker. The intra-oral mapping device may be integrated into the surgical implement and the second passive vectorized tracking marker may have a fixed three-dimensional spatial relationship with the intra-oral mapping device.

In a second embodiment the monitoring system may further comprise: a first passive vectorized tracking marker rigidly attached to the intra-oral mapping device in a predetermined relative fixed three-dimensional spatial position and orientation with respect to intra-oral mapping device; and a non-stereo optical tracker in data communication with the controller and configured and disposed for obtaining from a field of view of the tracker real time image information of at least the first tracking marker; the software program comprising a second series of instructions which when executed by the processor determines from the real time image information of the first tracking marker a current three-dimensional spatial position and orientation of the first tracking marker, and relates the scan data and the current intra-oral mapping information to the current three-dimensional spatial position and orientation of the first tracking marker.

In the same second embodiment, the monitoring system may further comprise a surgical implement bearing a second passive vectorized tracking marker in fixed three-dimensional spatial relationship with a working tip of the surgical implement and disposed within the field of view of the tracker, wherein the real time image information of at least the first tracking marker further includes information of the second tracking marker, and the software program comprises a series of instructions which when executed by the processor determines from the real time image information a current position and orientation of the second tracking marker, and relates a position and orientation of a working tip of the surgical implement to the surgical site based on the real time information of the second tracking marker.

As variant of the same embodiment, the monitoring system may comprise a surgical implement having a working tip, and wherein the surgical implement is integrated into the intra-oral mapping device; the first passive vectorized tracking marker has a fixed three-dimensional spatial relationship with the working tip of the surgical implement; and the software program comprises a third series of instructions which when executed by the processor determines from the real time information of the first tracking marker a current position and orientation of the first tracking marker and relates a position and orientation of the working tip of the surgical implement to the surgical site based on the real time information of the first tracking marker.

In a further aspect, a method is provided for superimposing three dimensional intra-oral mapping information on pre-surgical scan data, the method comprising: removably and rigidly attaching a single passive vectorized scan-visible fiducial reference proximate an oral surgical site of a surgical patient; performing a pre-surgical scan of the surgical site with the fiducial reference attached to obtain the scan data; obtaining from the scan data the three-dimensional spatial relationship between the fiducial reference and the surgical site; mapping by means of an intra-oral mapping device an intra-oral area of the patient including the surgical site and the fiducial reference to obtain mapping information about the surgical site and the fiducial reference; deriving from the mapping information a three-dimensional intra-oral map of the intra-oral area; determining from the mapping information the spatial location and orientation of the fiducial reference relative to the surgical site; and superimposing the intra-oral map on the pre-surgical scan data based on the spatial relationship between the fiducial reference and the surgical site in the scan data and the spatial relationship between the fiducial reference and the surgical site in the intra-oral map. The method may further comprise displaying the superimposed intra-oral map and the pre-surgical scan data on a display system. The mapping, deriving, determining, superimposing and displaying may be done in real time.

In a first extension of the method provided here, the method may further comprise rigidly and removably disposing a first passive vectorized tracking marker in a predetermined fixed three-dimensional spatial position and orientation relative to the single fiducial reference; operating a non-stereo optical tracker to gather real time image information of at least the first tracking marker; deriving from the real time image information of the first tracking marker a current three-dimensional spatial position and orientation of the first tracking marker; and relating the scan data and current intra-oral mapping information to the current three-dimensional spatial position and orientation of the first tracking marker.

The same first extension of the method may further comprise disposing within a field of view of the tracker a surgical implement bearing a second passive vectorized tracking marker in fixed three-dimensional spatial relationship with a working tip of the surgical implement; operating the tracker to gather real time image information of the second tracking marker; deriving from the real time image information a current position and orientation of the second tracking marker; and relating a position and orientation of a working tip of the surgical implement to the surgical site based on the real time information of the first and second tracking markers. The disposing a surgical instrument may comprise disposing surgical instrument wherein the intra-oral mapping device is integrated into surgical instrument, the intra-oral mapping device having a known fixed spatial relationship with the second passive vectorized tracking marker.

In a second extension of the method provided here, the method may further comprise operating a non-stereo optical tracker to obtain real time image information of at least a first tracking marker rigidly attached to the mapping device in a predetermined relative fixed three-dimensional spatial position and orientation with respect to the mapping device; deriving from the real time image information of the first tracking marker a current three-dimensional spatial position and orientation of the first tracking marker; relating the scan data and the current intra-oral mapping information to the current three-dimensional spatial position and orientation of the first tracking marker.

The same second extension of the method may comprise disposing within a field of view of the tracker a surgical implement bearing a second passive vectorized tracking marker in fixed three-dimensional spatial relationship with a working tip of the surgical implement; operating the non-stereo optical tracker to gather real time image information of the second tracking marker; deriving from the real time image information of the second tracking marker a current position and orientation of the second tracking marker; and relating a position and orientation of the working tip of the surgical implement to the surgical site based on the real time information of the second tracking marker.

The same second extension may instead comprise disposing within a field of view of the tracker a surgical implement integrated with the intra-oral mapping device, the first tracking marker having a known and fixed spatial relationship with a working tip of the surgical implement; and relating a position and orientation of the working tip of the surgical implement to the surgical site based on the real time information of the first tracking marker.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized as controller and display in conjunction with embodiments of the present invention.

FIGS. 3A-J are drawings of hardware components of the surgical monitoring system according to embodiments of the invention.

FIGS. 4A-C is a flowchart diagram illustrating one embodiment of the registering method of the present invention.

FIG. 5 is a drawing of a passive vectorized dental fiducial key with a tracking pole and a dental drill according to one embodiment of the present invention.

FIG. 6 is a drawing of an endoscopic surgical site showing the vectorized fiducial key, endoscope, and biopsy needle according to another embodiment of the invention.

FIG. 7 is a drawing of a three-dimensional position and orientation tracking system according to another embodiment of the present invention.

FIG. 8 is a drawing of a three-dimensional position and orientation tracking system according to yet another embodiment of the present invention.

FIG. 9 is a flowchart illustrating a method for monitoring a surgical site.

FIG. 10 is a drawing of an embodiment of a monitoring system according to the present invention.

FIG. 11 is a drawing of a first extension to the embodiment of FIG. 10

FIG. 12 is a drawing of a second extension to the embodiment of FIG. 10

FIG. 13a is flow chart of a method for superimposing an intra-oral map on pre-surgical scan data associated with a surgical site of a dental patient according to the present invention.

FIG. 13b is a flow chart of an extension to the method of FIG. 13a.

FIG. 13c is a flow chart of an extension to the method of FIG. 13a that differs in part from the extension in FIG. 13b.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

The detailed descriptions that follow are presented in part in terms of algorithms and symbolic representations of operations on data bits within a computer memory representing alphanumeric characters or other information. The hardware components are shown with particular shapes and relative orientations and sizes using particular scanning techniques, although in the general case one of ordinary skill recognizes that a variety of particular shapes and orientations and scanning methodologies may be used within the teaching of the present invention. A computer generally includes a processor for executing instructions and memory for storing instructions and data, including interfaces to obtain and process imaging data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities, observing and measuring scanned data representative of matter around the surgical site. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed to capture the underlying data of an image. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory, which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein that form part of the present invention; the operations are machine operations. Useful machines for performing the operations of the present invention include general-purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general-purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipments through signals configured to particular protocols, which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects. Often, but not necessarily, a physical object has a corresponding software object that may collect and transmit observed data from the physical device to the software system. Such observed data may be accessed from the physical object and/or the software object merely as an item of convenience; therefore where “actual data” is used in the following description, such “actual data” may be from the instrument itself or from the corresponding software object or module.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and here the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions, which may include sending additional messages to one or more other objects. The other objects may in turn carry out additional functions in response to the messages they receive. Including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms that are used frequently have specialized meanings in the present context. The term “object” relates to a set of computer instructions and associated data, which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers that are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information, which is formatted in a Standard Generalized Markup Language (“SGML”) or a HyperText Markup Language (“HTML”), both being scripting languages, which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular, or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system, which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 7. “Mobile Apps” refers to software programs written for execution with Mobile Software.

The terms “scan, fiducial reference”, “fiducial location”, “marker,” “tracker” and “image information” have particular meanings in the present disclosure. For purposes of the present disclosure, “scan” or derivatives thereof refer to x-ray, magnetic resonance imaging (MRI), computerized tomography (CT), sonography, cone beam computerized tomography (CBCT), or any system that produces a quantitative spatial representation of a patient and a “scanner” is the means by which such scans are obtained. The term “fiducial key”, or “fiducial reference”, or simply “fiducial” refers to an object or reference on the image of a scan that is uniquely identifiable as a fixed recognizable point. In the present specification the term “fiducial location” refers to a useful location to which a fiducial reference is attached. A “fiducial location” will typically be proximate a surgical site. The term “marker” or “tracking marker” refers to an object or reference that may be perceived by a sensor proximate to the location of the surgical or dental procedure, where the sensor may be an optical sensor, a radio frequency identifier (RFID), a sonic motion detector, an ultra-violet or infrared sensor. The term “tracker” refers to a device or system of devices able to determine the location of the markers and their orientation and movement continually in ‘real time’ during a procedure. As an example of a possible implementation, if the markers are composed of printed targets then the tracker may include a stereo camera pair. In some embodiments, the tracker may be a non-stereo optical tracker, for example a camera. The camera may, for example, operate in the visible or near-infrared range. The term “image information” is used in the present specification to describe information obtained by the tracker, whether optical or otherwise, and usable for determining the location of the markers and their orientation and movement continually in ‘real time’ during a procedure. In some embodiments, an imaging device may be employed to obtain real time close-up images of the surgical site quite apart from the tracker. In this specification, such imaging devices are described by the term “in situ imager” and the in situ imager may comprise an “illuminator” and an “imaging sensor”. The term “vectorized” is used in this specification to describe fiducial keys and tracking markers that are at least one of shaped and marked so as to make their orientation in three dimensions uniquely determinable from their appearance in a scan or in image information. If their three-dimensional orientation is determinable, then their three-dimensional location is also known.

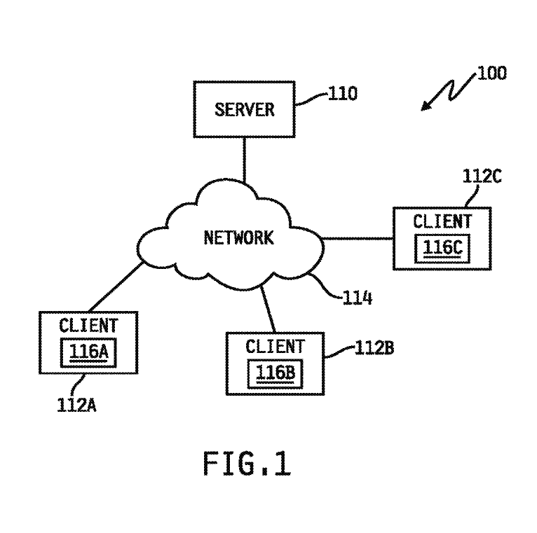

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

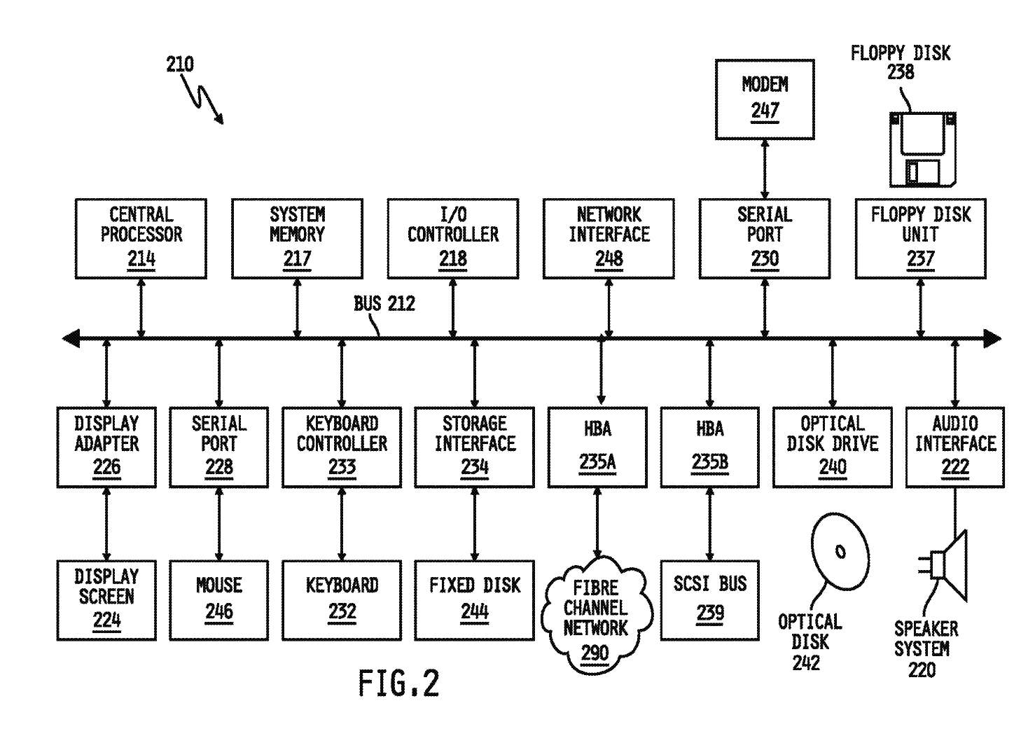

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fiber Channel network 290, host bus adapter (HBA) interface card 235B operative to connect to SCSI bus 239, and optical disk drive 240 operative to receive optical disk 242. Also included are mouse 246 (or other point-and-click device. coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS), which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an Internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

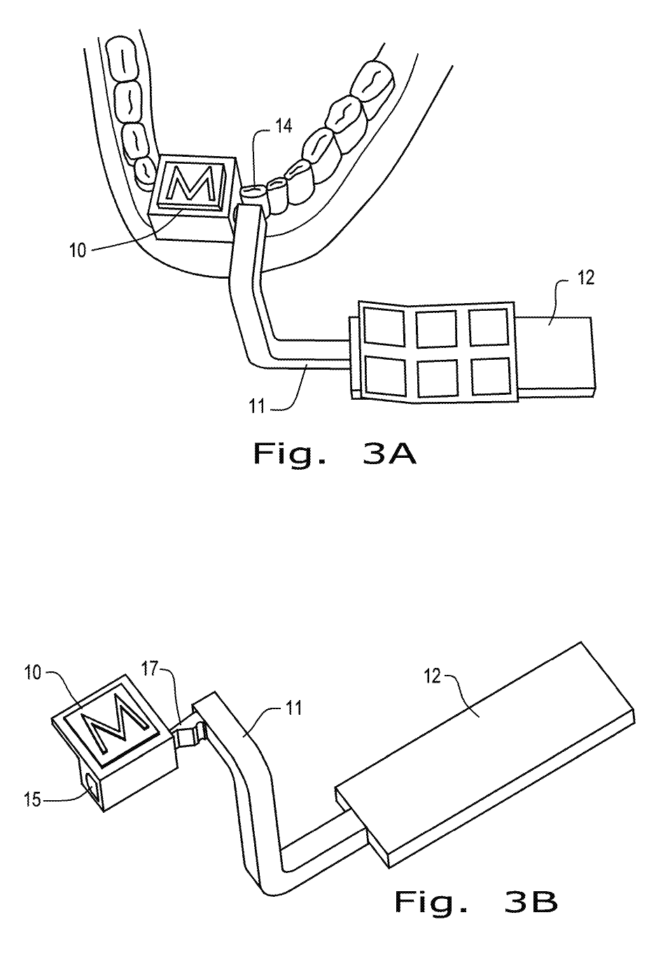

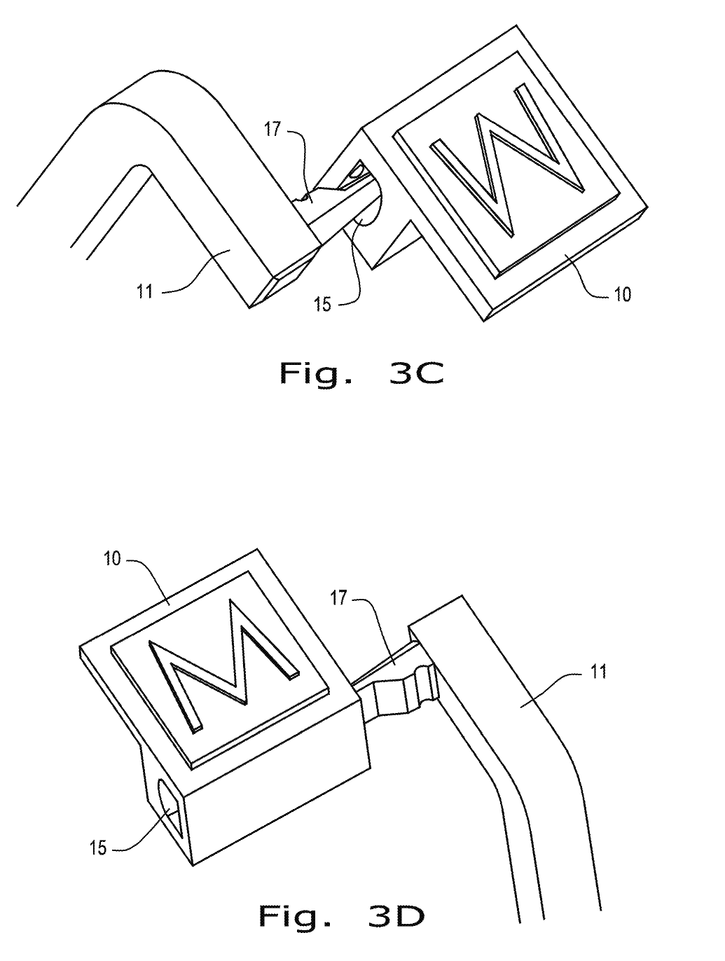

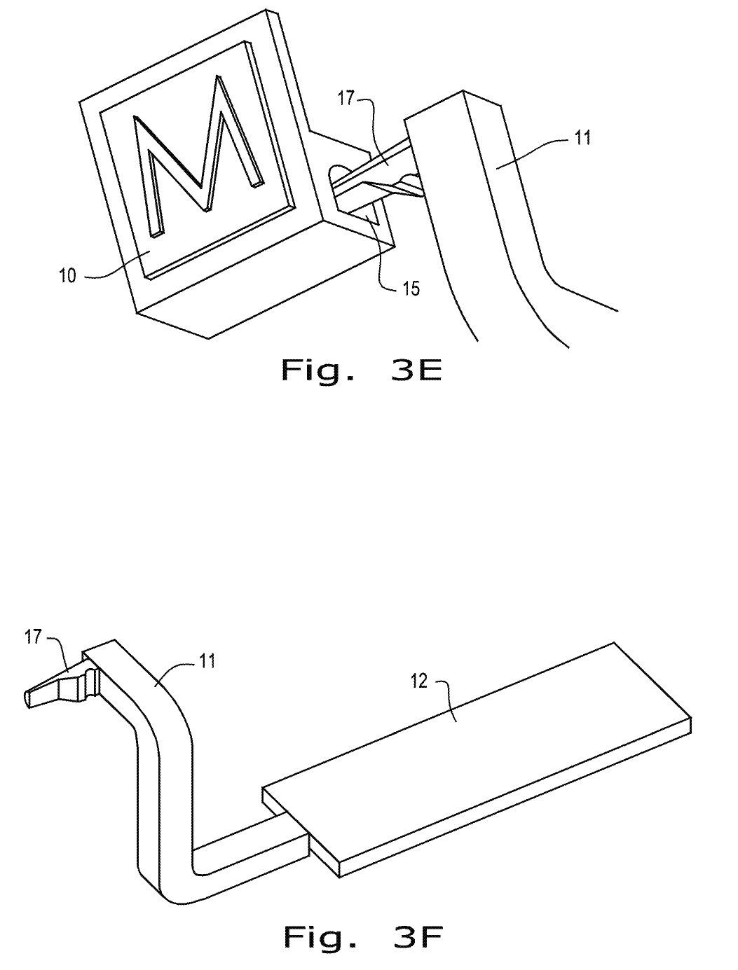

Many other devices or subsystems (not shown) may be connected in a similar manner (e. g., document scanners, digital cameras and so on), including the hardware components of FIGS. 3A-M, which alternatively may be in communication with associated computational resources through local, wide-area, or wireless networks or communications systems. Thus, while the disclosure may generally discuss an embodiment where the hardware components are directly connected to computing resources, one of ordinary skill in this area recognizes that such hardware may be remotely connected with computing resources. Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNLX is a registered trademark of X/Open Company Limited of Reading, United Kingdom), Linux® (Linux is a registered trademark of Linus Torvalds of Portland, Oreg.), or other known or developed operating system.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modification to the first signal, whether due to circuit limitations or due to passage through other circuit elements which do not change the informational and/or final functional aspect of the first signal.

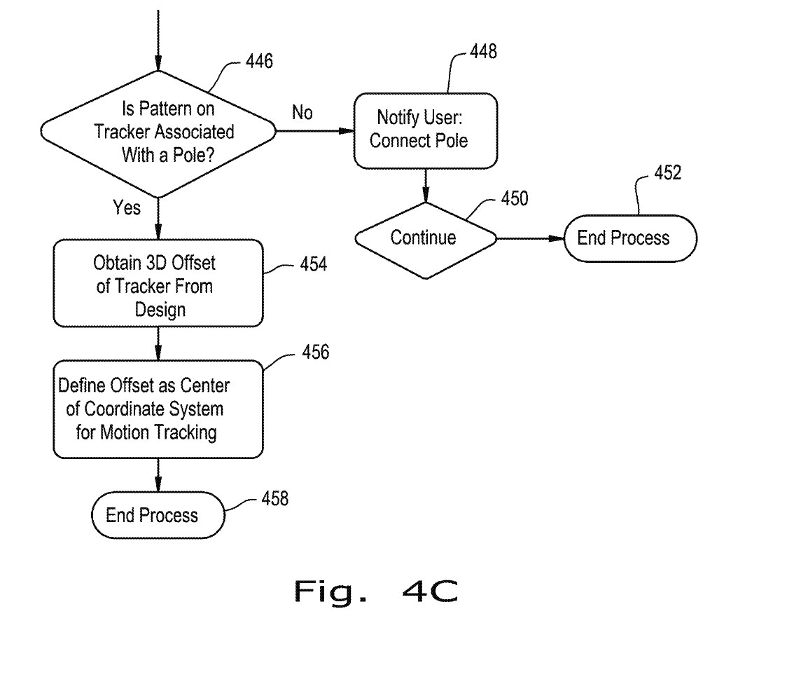

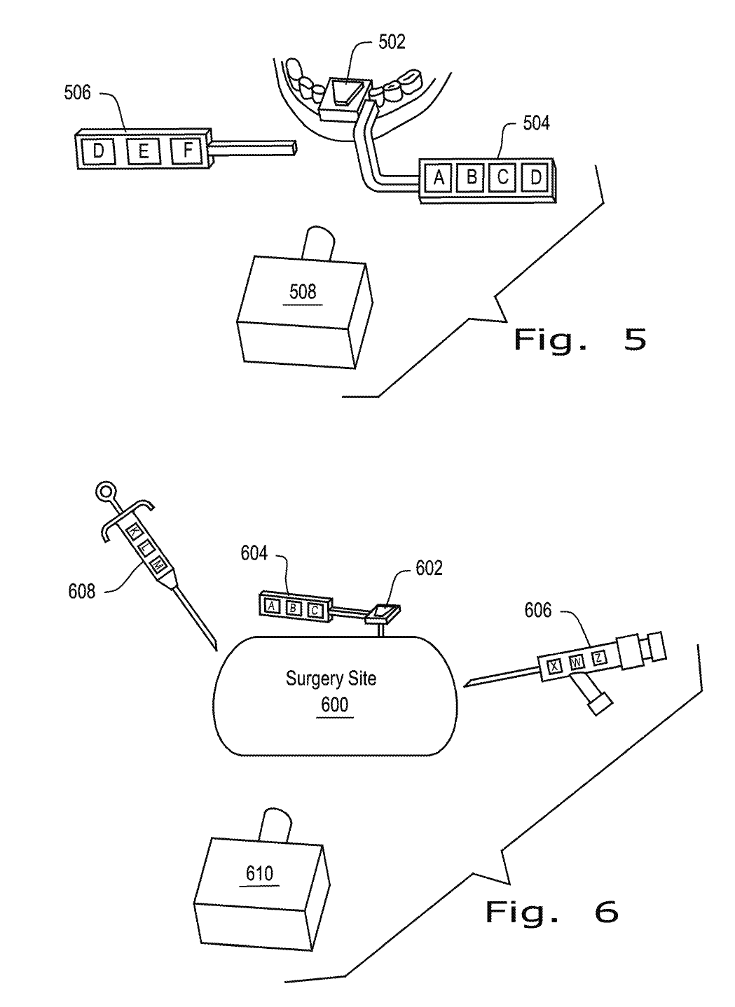

The present invention relates to embodiments of surgical hardware and software monitoring systems and methods which allow for surgical planning while the patient is available for surgery, for example while the patient is being prepared for surgery so that the system may model the surgical site. The system uses a particularly configured piece of hardware, namely a vectorized fiducial reference, represented as fiducial key 10 in FIG. 3A, to orient vectorized tracking marker 12 of the monitoring system with regard to the critical area of the surgery. Single fiducial key 10 is attached to a location near the intended surgical area, in the exemplary embodiment of the dental surgical area of FIG. 3A, fiducial key 10 is attached to a dental splint 14. Vectorized tracking marker 12 may be connected to fiducial key 10 by tracking pole 11. In embodiments in which the fiducial reference is directly visible to a suitable tracker (see for example FIG. 5 and FIG. 6) that acquires image information about the surgical site, a tracking marker may be attached directly to the fiducial reference. The tracker may be a non-stereo optical tracker. For example, in a dental surgical procedure, the dental tracking marker 14 may be used to securely locate the fiducial 10 near the surgical area. The single fiducial key 10 may be used as a point of reference, or a fiducial, for the further image processing of data acquired from tracking marker 12 by the tracker. In this arrangement, fiducial key or reference 10 is scanned not by the tracker, which may for example be an optical tracker, but by a suitable scanning means, which may for example be an X-ray system, CAT scan system, or MRI system as per the definition of “scan” above. In some applications, fiducial key 10 may be disposed in a location or in such orientation as to be at least in part non-visible to the tracker of the system.

In other embodiments additional vectorized tracking markers 12 may be attached to items independent of the fiducial key 10 and any of its associated tracking poles 11 or tracking markers 12. This allows the independent items to be tracked by the tracker.

In a further embodiment at least one of the items or instruments near the surgical site may optionally have a tracker attached to function as tracker for the monitoring system of the invention and to thereby sense the orientation and the position of the tracking marker 12 and of any other additional vectorized tracking markers relative to the scan data of the surgical area. By way of example, the tracker attached to an instrument may be a miniature digital camera and it may be attached, for example, to a dentist's drill. Any other vectorized markers to be tracked by the tracker attached to the item or instrument must be within the field of view of the tracker.

Using the dental surgery example, the patient is scanned to obtain an initial scan of the surgical site. The particular configuration of single fiducial key 10 allows computer software stored in memory and executed in a suitable controller, for example processor 214 and memory 217 of computer 210 of FIG. 2, to recognize its relative position within the surgical site from the scan data, so that further observations may be made with reference to both the location and orientation of fiducial key 10. In some embodiments, the fiducial reference includes a marking that is apparent as a recognizable identifying symbol when scanned. In other embodiments, the fiducial reference includes a shape that is distinct in the sense that the body apparent on the scan has an asymmetrical form allowing the front, rear, upper, and lower, and left/right defined surfaces that may be unambiguously determined from the analysis of the scan, thereby to allow the determination not only of the location of the fiducial reference, but also of its orientation. That is, the shape and/or markings of the fiducial reference render it vectorized. The marking and/or shape of fiducial key 10 allows it to be used as the single and only fiducial key employed in the surgical hardware and software monitoring system. By comparison, prior art systems typically rely on a plurality of fiducials. Hence, while the tracker may track several vectorized tracking markers within the monitoring system, only a single vectorized fiducial reference or key 10 of known shape or marking is required. By way of example, FIG. 5, later discussed in more detail, shows vectorized markers 506 and 504 tracked by tracker 508, but there is only one vectorized fiducial reference or key 502 in the system. FIG. 6 similarly shows three vectorized markers 604, 606, and 608 being tracked by tracker 610, while there is only a single vectorized fiducial reference or key 602 in the system.

In addition, the computer software may create a coordinate system for organizing objects in the scan, such as teeth, jaw bone, skin and gum tissue, other surgical instruments, etc. The coordinate system relates the images on the scan to the space around the fiducial and locates the instruments bearing markers both by orientation and position. The model generated by the monitoring system may then be used to check boundary conditions, and in conjunction with the tracker display the arrangement in real time on a suitable display, for example display 224 of FIG. 2.

In one embodiment, the computer system has a predetermined knowledge of the physical configuration of single fiducial key 10 and examines slices/sections of the scan to locate fiducial key 10. Locating of fiducial key 10 may be on the basis of its distinct shape, or on the basis of distinctive identifying and orienting markings upon the fiducial key or on attachments to the fiducial key 10 such as tracking marker 12. Fiducial key 10 may be rendered distinctly visible in the scans through higher imaging contrast by the employ of radio-opaque materials or high-density materials in the construction of the fiducial key 10. In other embodiments the material of the distinctive identifying and orienting markings may be created using suitable high density or radio-opaque inks or materials. In the present specification, the term “scan-visible” is used to describe the characteristic of fiducial key 10 by which it is rendered visible in a scan, while not necessarily otherwise visible to the human eye or optical sensor.

Once fiducial key 10 is identified, the location and orientation of the fiducial key 10 is determined from the scan segments, and a point within fiducial key 10 is assigned as the center of the coordinate system. The point so chosen may be chosen arbitrarily, or the choice may be based on some useful criterion. A model is then derived in the form of a transformation matrix to relate the fiducial system, being fiducial key 10 in one particular embodiment, to the coordinate system of the surgical site. The resulting virtual construct may be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

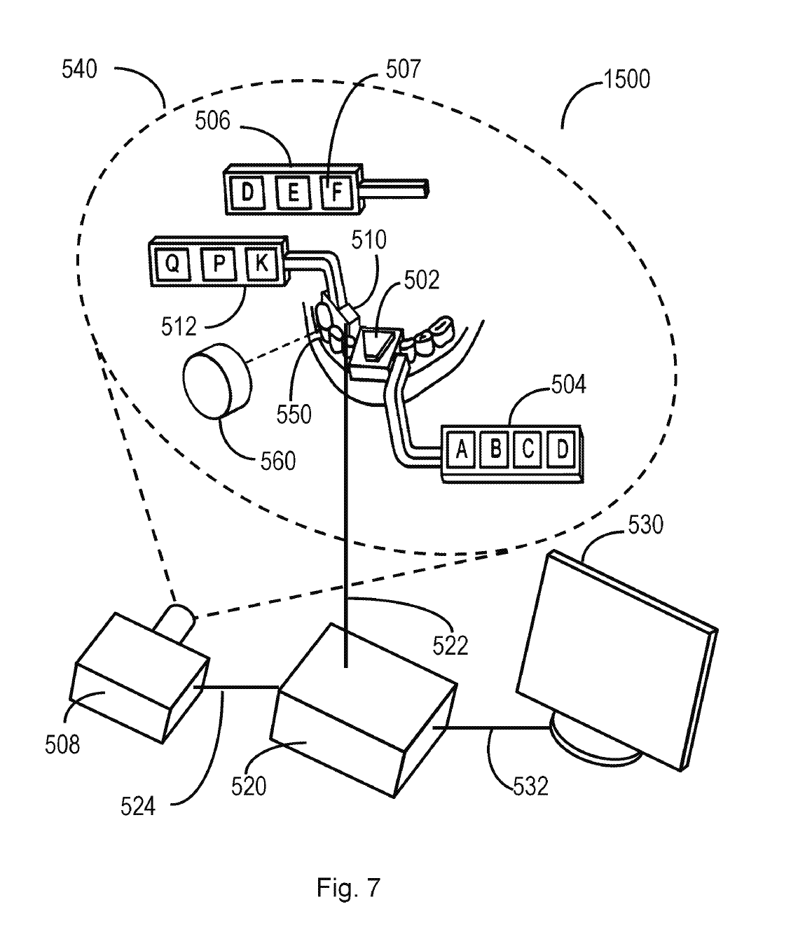

In some embodiments, the monitoring hardware includes a tracking attachment to the fiducial reference. In the embodiment pertaining to dental surgery the tracking attachment to fiducial key 10 is tracking marker 12, which is attached to fiducial key 10 via tracking pole 11. Tracking marker 12 may have a particular identifying pattern, described in more detail later at the hand of FIGS. 7-10. The trackable attachment, for example tracking marker 12, and even associated tracking pole 11 may have known configurations so that observational data from tracking pole 11 and/or tracking marker 12 may be precisely mapped to the coordinate system, and thus progress of the surgical procedure may be monitored and recorded. For example, as particularly shown in FIG. 3J, fiducial key 10 may have hole 15 in a predetermined location specially adapted for engagement with insert 17 of tracking pole 11. In such an arrangement, for example, tracking poles 11 may be attached with a low force push into hole 15 of fiducial key 10, and an audible haptic notification may thus be given upon successful completion of the attachment.

It is further possible to reorient the tracking pole during a surgical procedure. Such reorientation may be in order to change the location of the procedure, for example where a dental surgery deals with teeth on the opposite side of the mouth, where a surgeon switches hands, and/or where a second surgeon performs a portion of the procedure. For example, the movement of the tracking pole may trigger a re-registration of the tracking pole with relation to the coordinate system, so that the locations may be accordingly adjusted. Such a re-registration may be automatically initiated when, for example in the case of the dental surgery embodiment, tracking pole 11 With its attached tracking marker 12 are removed from hole 15 of fiducial key 10 and another tracking marker with its associated tracking pole is connected to an alternative hole on fiducial key 10. Additionally, boundary conditions may be implemented in the software so that the user is notified when observational data approaches and/or enters the boundary areas.

In a further embodiment, the tracking markers may specifically have a three dimensional shape. Suitable three-dimensional shapes bearing identifying patterns may include, without limitation, a segment of an ellipsoid surface and a segment of a cylindrical surface. In general, suitable three-dimensional shapes are shapes that are mathematically describable by simple functions.

In a further embodiment of the system utilizing the invention, a surgical instrument or implement, herein termed a “hand piece” (see FIGS. 5 and 6), may also have a particular configuration that may be located and tracked in the coordinate system and may have suitable tracking markers as described herein. A boundary condition may be set up to indicate a potential collision with virtual material, so that when the hand piece is sensed to approach the boundary condition an indication may appear on a screen, or an alarm sound. Further, target boundary conditions may be set up to indicate the desired surgical area, so that when the trajectory of the hand piece is trending outside the target area an indication may appear on screen or an alarm sound indicating that the hand piece is deviating from its desired path.

An alternative embodiment of some hardware components are shown in FIGS. 3G-I. Fiducial key 10′ has connection elements with suitable connecting portions to allow a tracking pole 11′ to position a tracking marker 12′ relative to the surgical site. Conceptually, fiducial key 10′ serves as an anchor for pole 11′ and tracking marker 12′ in much the same way as the earlier embodiment, although it has a distinct shape. The software of the monitoring system is pre-programmed with the configuration of each particularly identified fiducial key, tracking pole, and tracking marker, so that the location calculations are only changed according to the changed configuration parameters.

The materials of the hardware components may vary according to regulatory requirements and practical considerations. Generally, the key or fiducial component is made of generally radio opaque material such that it does not produce noise for the scan, yet creates recognizable contrast on the scanned image so that any identifying pattern associated with it may be recognized. In addition, because it is generally located on the patient, the material should be lightweight and suitable for connection to an apparatus on the patient. For example, in the dental surgery example, the materials of the fiducial key must be suitable for connection to a plastic splint and suitable for connection to a tracking pole. In the surgical example the materials of the fiducial key may be suitable for attachment to the skin or other particular tissue of a patient.

The vectorized tracking markers may be clearly identified by employing, for example without limitation, high contrast pattern engraving. The materials of the tracking markers are chosen to be capable of resisting damage in autoclave processes and are compatible with rigid, repeatable, and quick connection to a connector structure. The tracking markers and associated tracking poles have the ability to be accommodated at different locations for different surgery locations, and, like the fiducial keys, they should also be relatively lightweight as they will often be resting on or against the patient. The tracking poles must similarly be compatible with autoclave processes and have connectors of a form shared among tracking poles.

The tracker employed in tracking the fiducial keys, tracking poles and tracking markers should be capable of tracking with suitable accuracy objects of a size of the order of 1.5 square centimeters. The tracker may be, by way of example without limitation, a stereo camera or stereo camera pair. While the tracker is generally connected by wire to a computing device to read the sensory input, it may optionally have wireless connectivity to transmit the sensory data to a computing device. In other embodiments, the tracker may be a non-stereo optical tracker.

In embodiments that additionally employ a trackable piece of instrumentation, such as a hand piece, vectorized tracking markers attached to such a trackable piece of instrumentation may also be light-weight; capable of operating in a 3 object array with 90 degrees relationship; optionally having a high contrast pattern engraving and a rigid, quick mounting mechanism to a standard hand piece.

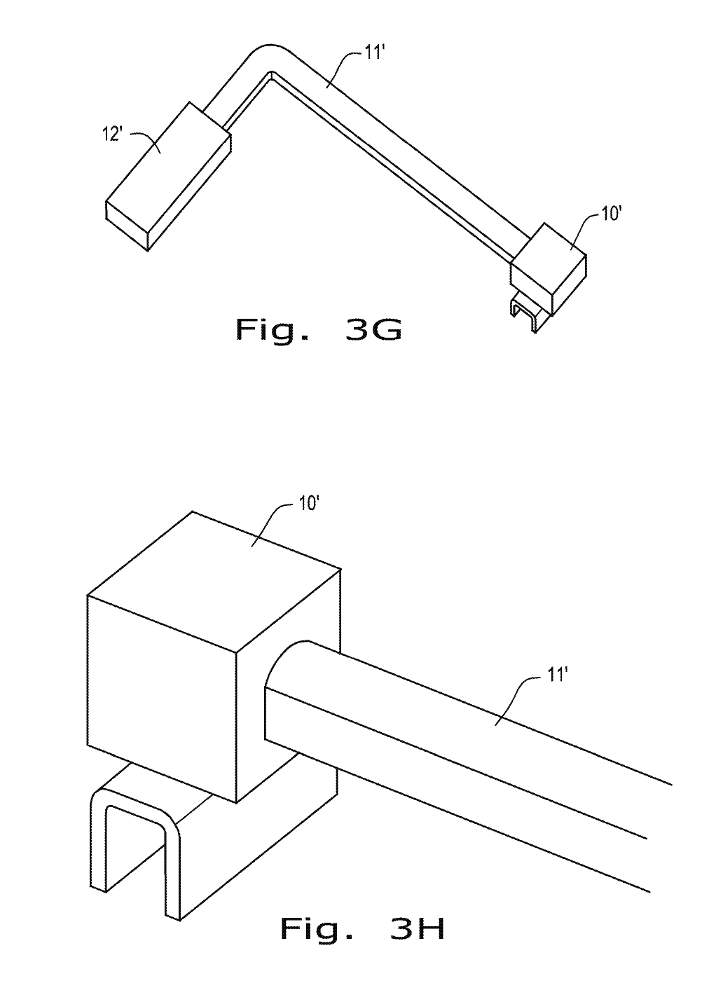

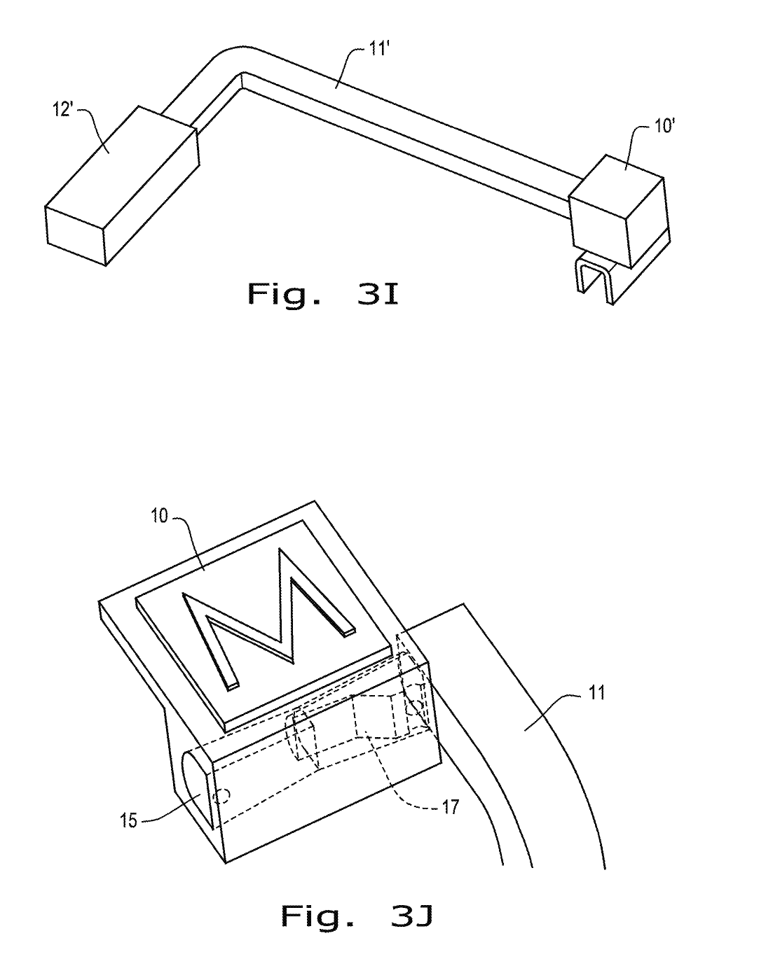

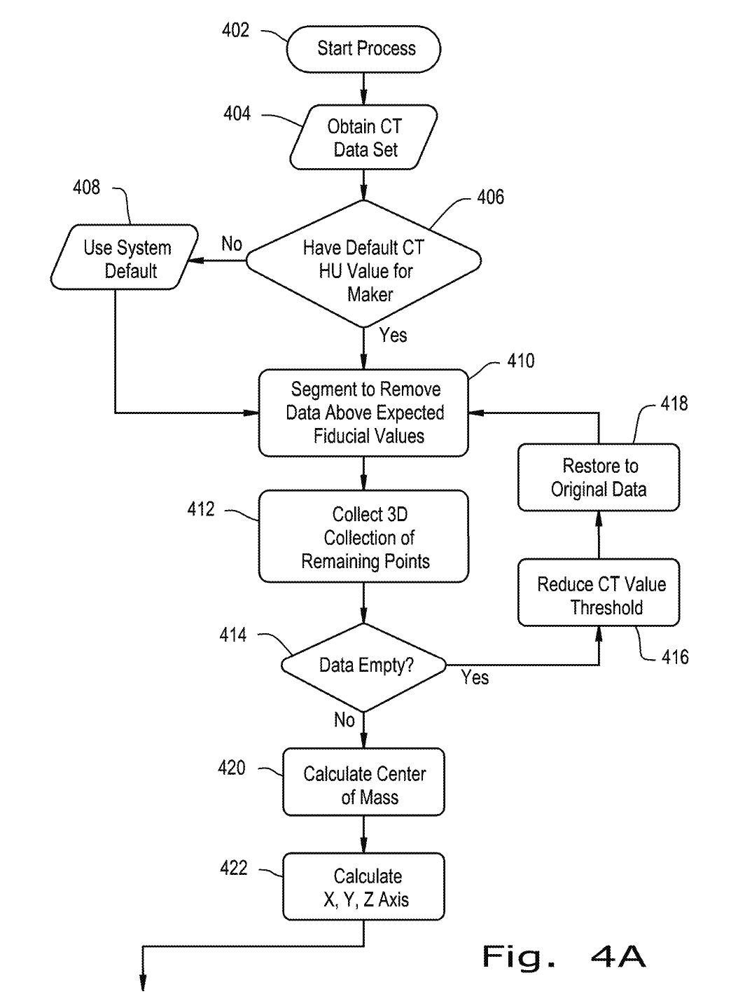

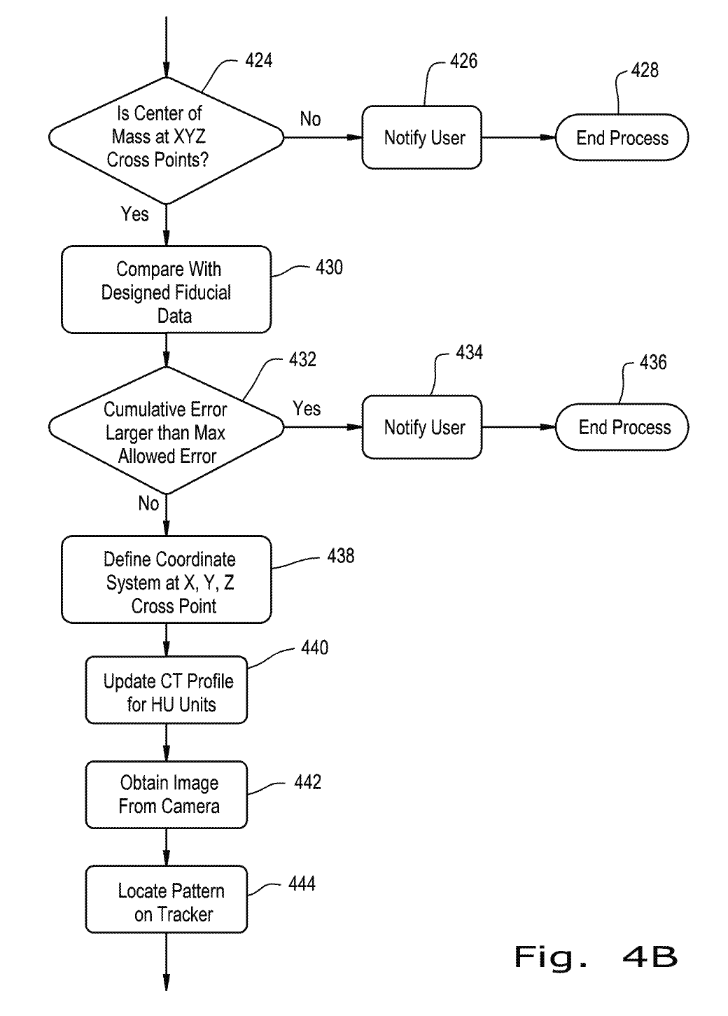

In another aspect there is presented an automatic registration method for tracking surgical activity, as illustrated in FIGS. 4A-C. FIG. 4A and FIG. 4B together present, without limitation, a flowchart of one method for determining the three-dimensional location and orientation of the fiducial reference from scan data. FIG. 4C presents a flow chart of a method for confirming the presence of a suitable tracking marker in image information obtained by the tracker and determining the three-dimensional location and orientation of the fiducial reference based on the image information.

Once the process starts [402], as described in FIGS. 4A and 4B, the system obtains a scan data set [404] from, for example, a CT scanner and checks for a default CT scan Hounsfield unit (HU) value [at 406] for the vectorized fiducial which may or may not have been provided with the scan based on a knowledge of the fiducial and the particular scanner model, and if such a threshold value is not present, then a generalized predetermined default value is employed [408]. Next the data is processed by removing scan segments with Hounsfield data values outside expected values associated with the fiducial key values [at 410], following the collection of the remaining points [at 412]. If the data is empty [at 414], the CT value threshold is adjusted [at 416], the original value restored [at 418], and the segmenting processing scan segments continues [at 410]. Otherwise, with the existing data a center of mass is calculated [at 420], along with calculating the X, Y, and Z axes [at 422]. If the center of mass is not at the cross point of the XYZ axes [at 424], then the user is notified [at 426] and the process stopped [at 428]. If the center of mass is at the XYZ cross point then the data points are compared with the designed fiducial data [430]. If the cumulative error is larger than the maximum allowed error [432] then the user is notified [at 434] and the process ends [at 436]. If not, then the coordinate system is defined at the XYZ cross point [at 438], and the scan profile is updated for the HU units [at 440].

Turning now to FIG. 4C, image information is obtained from the tracker, being a suitable camera or other sensor [442]. The image information is analyzed [444] to determine whether a tracking marker is present in the image information. If not, then the user is queried [446] as to whether the process should continue or not. If not, then the process is ended [448]. If the process is to continue, then the user may be notified [450] that no tracking marker has been found in the image information, and the process returns to obtaining image information [442]. If a tracking marker has been found based on the image information, or one has been attached by the user upon the above notification [at 450], the offset and relative orientation of the tracking marker to the fiducial reference is obtained [452] from a suitable database. The term “database” is used in this specification to describe any source, amount or arrangement of such information, whether organized into a formal multi-element or multi-dimensional database or not. Such a database may be stored, for example, in system memory 217, fixed disk 244, or in external memory through network interface 248. A single data set comprising offset value and relative orientation may suffice in a simple implementation of this embodiment of the invention and may be provided, for example, by the user or may be within a memory unit of the controller or in a separate database or memory.

The offset and relative orientation of the tracking marker is used to define the origin of a coordinate system at the fiducial reference and to determine the three-dimensional orientation of the fiducial reference based on the image information [454] and the registration process ends [458]. In order to monitor the location and orientation of the fiducial reference in real time, the process may be looped back from step [454] to obtain new image information from the camera [442]. A suitable query point may be included to allow the user to terminate the process. Detailed methods for determining orientations and locations of predetermined shapes or marked tracking markers from image data are known to practitioners of the art and will not be dwelt upon here. The coordinate system so derived is then used for tracking the motion of any items bearing vectorized tracking markers in the proximity of the surgical site. Other registration systems are also contemplated, for example using current other sensory data rather than the predetermined offset, or having a fiducial with a transmission capacity.

One example of an embodiment of the invention is shown in FIG. 5. In addition to vectorized fiducial key 502 mounted at a predetermined tooth and having a rigidly mounted vectorized tracking marker 504, an additional instrument or implement 506, for example a hand piece which may be a dental drill, may be observed by a camera 508 serving as tracker of the monitoring system.

Another example of an embodiment of the invention is shown in FIG. 6. Surgery site 600, for example a human stomach or chest, may have fiducial key 602 fixed to a predetermined position to support tracking marker 604. Other apparatus with suitable tracking markers may be in use in the process of the surgery at surgery site 600. By way of non-limiting example, endoscope 606 may have a further vectorized tracking marker, and biopsy needle 608 may also be present bearing a vectorized tracking marker at surgery site 600. Sensor 610, serving as tracker for the system, may be for example a camera, infrared sensing device, or RADAR. In particular, the tracker may be a two-dimensional imaging tracker that produces a two dimensional image of the surgery site 600 for use as image information for the purposes of embodiments of the invention, including two dimensional image information of any vectorized tracking markers in the field of view of the tracker. The camera may be, for example, a non-stereo optical camera. Surgery site 600, endoscope 606, biopsy needle 608, fiducial key 602 and vectorized tracking marker 604 may all be in the field of view of tracker 610.

The trackers 508,610 of the systems and methods of the present invention may comprise a single optical imager obtaining a two-dimensional image of the site being monitored. The system and method described in the present specification allow three-dimensional locations and orientations of tracking markers to be obtained using non-stereo-pair two-dimensional imagery. In some embodiments more than one imager may be employed as tracker, but the image information required and employed is nevertheless two-dimensional. Therefore the two imagers may merely be employed to secure different perspective views of the site, each imager rendering a two-dimensional image that is not part of a stereo pair. This does not exclude the employment of stereo-imagers in obtaining the image information about the site, but the systems and methods of the present invention are not reliant on stereo imagery of the site in order to identify and track any of the passive vectorized tracking markers employed in the present invention. By virtue of their shapes or markings, the three-dimensional locations and orientations of the tracking markers may be completely determined from a single two-dimensional image of the field of view of the tracker.

All vectorized tracking markers employed in the present invention may be passive. The term “passive” is used in the present specification to describe markers that do not rely on any own electronic, electrical, optoelectronic, optical, magnetic, wireless, inductive, or other active signaling function or on any incorporated electronic circuit, whether powered or unpowered, to be identified, located, or tracked. The term “own active signaling” is used in this specification to describe a signal that is temporally modulated by, on, or within the tracking marker. The tracking markers do not rely on motion, location, or orientation sensing devices, whether powered or unpowered, to be tracked. They cannot sense their own motion, location, or orientation, nor have they any ability to actively communicate. They bear distinctive markings and/or have distinctive shapes that allow them to be identified, located, and tracked in three dimensions by a separate tracker such as, for example without limitation, tracker 610 of FIG. 6 or tracker 508 of FIGS. 5, 7, 8, 11 and 12, both in their location and in their orientation. In some embodiments, the tracker may be an optical tracker, more particularly, a non-stereo optical tracker. Any one or more of identification, location, and tracking of the markers is solely on the basis of their distinctive markings and/or distinctive shapes. All fiducial references described in the present specification, may also be passive. This specifically includes fiducial references 10 and 10′ in FIGS. 3A to 3J, key or fiducial reference 502 of FIGS. 5, 7, 8, 10, 11 and 12, and fiducial reference 602 of FIG. 6.

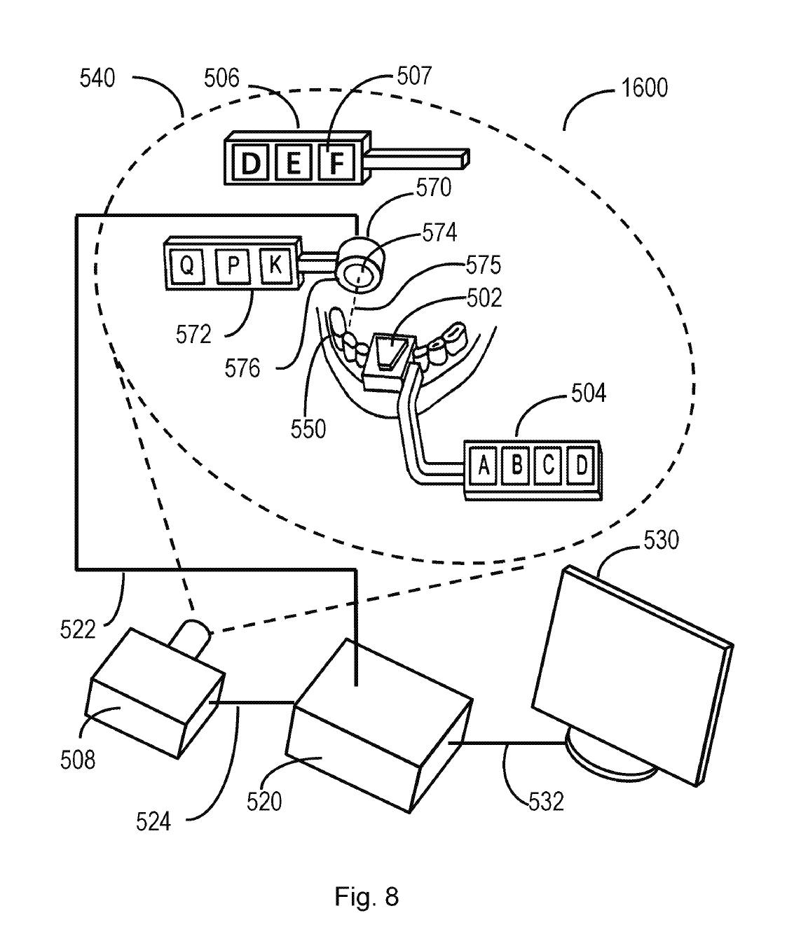

In another aspect of the invention there is provided a method, described with reference to FIG. 8, for relating in real time the three-dimensional location and orientation of surgical site 550 on a patient to the location and orientation of the surgical site in a scan of surgical site 550, the method comprising removably attaching single vectorized fiducial reference 502 to a fiducial location on the patient proximate surgical site 550; performing the scan with single fiducial reference 502 attached to the fiducial location to obtain scan data; determining the three-dimensional location and orientation of the fiducial reference from the scan data; obtaining real time image information of surgical site 550 (using tracker 508); determining in real time the three-dimensional location and orientation of single fiducial reference 502 from the image information; deriving a spatial transformation matrix or expressing in real time the three-dimensional location and orientation of the fiducial reference as determined from the image information in terms of the three-dimensional location and orientation of single fiducial reference 502 as determined from the scan data.

Obtaining of real time image information from surgical site 550 may comprise rigidly and removably attaching to single vectorized fiducial reference 502 first vectorized tracking marker 504 in a fixed three-dimensional spatial relationship with single fiducial reference 502. First tracking marker 504 may be configured for having its location and its orientation determined based on the image information. Attaching first tracking marker 504 to single fiducial reference 502 may comprise rigidly and removably attaching first tracking marker 504 to the fiducial reference by means of a tracking pole. In this regard, see for example tracking pole 11 of FIG. 3B used to attach vectorized tracking marker 12 to fiducial reference 10. Obtaining the real time image information of the surgical site may comprise rigidly and removably attaching to the fiducial reference a tracking pole in a fixed three-dimensional spatial relationship with the fiducial reference, and the tracking pole may have a distinctly identifiable three-dimensional shape that allows its location and orientation to be uniquely determined from the image information.

In yet a further aspect of the invention there is provided a method for real time monitoring the position of an object, for example object 506 in FIG. 8, in relation to surgical site 550 of a patient, the method comprising removably attaching single fiducial reference 502 to a fiducial location on the patient proximate surgical site 550; performing a scan with single fiducial reference 502 attached to the fiducial location to obtain scan data; determining the three-dimensional location and orientation of single fiducial reference 502 from the scan data; obtaining real time image information of surgical site 550 (using tracker 508); determining in real time the three-dimensional location and orientation of single fiducial reference 502 from the image information; deriving a spatial transformation matrix for expressing in real time the three-dimensional location and orientation of single fiducial reference 502 as determined from the image information in terms of the three-dimensional location and orientation of single fiducial reference 502 as determined from the scan data; determining in real time the three-dimensional location and orientation of object 506 from the image information; and relating the three-dimensional location and orientation of object 506 to the three-dimensional location and orientation of the fiducial reference as determined from the image information. Determining in real time the three-dimensional location and orientation of the object from the image information may comprise rigidly attaching second tracking marker 507 to object 506.

A further embodiment is shown schematically (and not to scale) in FIG. 7, which is based on the elements already described at the hand of the dental surgery example of FIG. 5. Three-dimensional position and orientation tracking system 1500 comprises X-ray imaging sensor 510 bearing passive vectorized tracking marker 512. Tracking marker 512 is disposed within field of view 540 of tracker 508, with X-ray imaging sensor 510 disposed to obtain live X-ray images of surgical site 550 during a surgical procedure. These live X-ray images may be obtained on a continuous basis, or may consist of a continuous series of individual snapshots. Tracking marker 512 is rigidly attached either directly or indirectly to X-ray imaging sensor 510 in a predetermined fixed location on X-ray imaging sensor 510 and at a predetermined fixed orientation relative to the viewing axis of X-ray imaging sensor 510, given by a broken straight line in FIG. 15. X-ray imaging sensor 510 is served by a suitable X-ray source 560 illuminating the surgical site 550 with X-rays.

System tracker 508 obtains image information of the region within field of view 540 of system tracker 508. The image information is provided to system controller 520 by tracker 508 via tracker data link 524. In FIG. 7, tracker data link 524 is shown as a wired link, but in other embodiments tracker data link 524 may involve radio, optical, or other suitable wireless link. System controller 520 is programmable with software configuring it for extracting from the image information the 3D location and orientation information of passive vectorized tracking markers 504 and 512 by the methods already described in detail above at the hand of FIGS. 1 to 6.

The 3D location and orientation information of tracking marker 504 allows system controller 520 to directly compute the 3D location and orientation of fiducial reference 502. Since fiducial reference 502 is rigidly attached to surgical site 550 in a known relative 3D location and orientation relationship, system controller 520 may thereby compute the 3D location and orientation of surgical site 550.

The 3D location and orientation information of tracking marker 512 allows system controller 520 to directly compute the 3D location and orientation of X-ray imaging sensor 510. This allows system controller 520 to track in real time the 3D location and orientational view obtained by X-ray imaging sensor 510.

When surgical site 550 is illuminated with X-rays by X-ray source 560, system controller 520 may directly relate X-ray images of surgical site 550 received by system controller 520 via X-ray sensor data link 522 to the 3D location and orientation information of surgical site 550. Controller 520 may display the result on monitor 530 via monitor link 532. Data links 522 and 532 are shown as wired in FIG. 7, but in other embodiments data links 522 and 532 may involve radio, optical, or other suitable wireless link. Data links 522 and 532 ensure that the controller 520 is data-wise coupled to X-ray imaging sensor 510 and tracker 508 respectively.

The combination of the location and orientation information from tracking marker 504 and 3D-located and oriented live X-ray images from X-ray imaging sensor 510 allows the updating of information about surgical site 550 during the surgical procedure. This, in turn, allows a continuously updated 3D-based rendering of surgical site 550 on monitor or display system 530, via monitor data line 532, to assist in the surgical procedure. This allows monitor 530 to show during the surgical procedure the current live image of surgical site 550 in three-dimensional spatial relationship relative to the scan data. System 1500 determines from the scan data, the image information, and the live images a continuously updated 3-dimensional model of surgical site 550 overlaid with live imagery of surgical site 550.

As with the embodiment of FIG. 5, an additional instrument or implement 506, for example a hand piece that may be a dental drill, may be observed and tracked by tracker 508 of the monitoring system. To this end, implement 506 may bear third passive vectorized tracking marker 507. As already explained at the hand of FIG. 6, the same arrangement may also be applied to non-dental surgery.

In the embodiment described above at the hand of FIG. 7, illuminator 560 may also have a passive vectorized tracking marker (not shown in the interest of clarity) fixedly attached in a fixed three-dimensional location and orientation relative to illuminator 560. Given this known fixed 3D relationship, a knowledge of the illumination cone of illuminator 560 allows the user to know where the illumination will be impinging once the location and orientation of the vectorized tracking marker on illuminator 560 is known. With illuminator 560 disposed in field of view 540 of tracker 508, system controller 520 may extract from the image information provided by tracker 508 the three-dimensional location and orientation of the tracking marker attached to illuminator 560 and display on monitor 530 an indication of where illuminator 560 will illuminate the patient at any given time. This allows the user to adjust the positioning of illuminator 560 proximate surgical site 550.

Another embodiment is described at the hand of FIG. 8. Every element of FIG. 8 bearing the same number as in FIG. 7 is to be understood as being the same element and performing the same function as in FIG. 7. In the embodiment of monitoring system 1600 shown in FIG. 8, in situ imager 570 comprises imaging sensor 574 for imaging surgical site 550 and illuminator 576 for illuminating surgical site 550 with radiation. Illuminator 576 may employ visible light radiation allowing imaging sensor 574 to image surgical site 550. In some implementations, illuminator 576 may employ exciting radiation, for example without limitation blue light, ultra-violet light, or other exciting radiation for exciting tissue to selectively fluoresce and emit light of a longer or shorter wavelength. Imaging sensor 574 may be an imaging sensor sensitive to the illuminating radiation from illuminator 576. In some implementations, illuminator 576 may be an annular illuminator disposed around imaging sensor 574. In other implementations, illuminator 576 and imaging sensor 574 may be separate devices, with imaging sensor 574 directly or indirectly bearing the rigidly attached tracking sensor 572.

When exciting radiation from illuminator 576 is employed to induce fluorescence in the tissue of surgical site 550, imaging sensor may be sensitive to the induced fluorescence light wavelengths and may be rendered specifically insensitive to the exciting radiation wavelength by means of suitable optical filters. In yet other implementations, in situ imager 570 may be equipped with both visible imaging facilities and fluorescence imaging facilities in order to superimpose the fluorescence image on the visible image. In yet other implementations the illuminating radiation may be of one spectrum of wavelengths while the imaging sensor 574 employs a different spectrum chosen to improve imaging contrast within imaging sensor 574.

Passive vectorized tracking marker 572 is attached directly or indirectly to imaging sensor 574 in a predetermined fixed location with respect to imaging sensor 574 and at a predetermined fixed orientation relative to the viewing axis of imaging sensor 574, given by broken straight line 575 in FIG. 8. System controller 520 receives live images of the surgical site over sensor data link 526 which ensures that controller 520 is data-wise coupled to imaging sensor 574. The embodiment of FIG. 8 therefore differs from the embodiment of FIG. 7 in that the means of imaging is reflective or fluoroscopic, while the means of imaging in FIG. 7 is X-ray transmissive. In both embodiments illuminator 560, 576 is employed and in both embodiments a live image, being either continuously generated images or comprising intermittent snapshots, is obtained of the surgical site 550 by an imaging sensor 510, 574. In both cases the live image of surgical site 550 is communicated to system controller 520 via sensor data link 522, 526. The live images may be one or more of reflected visible light images, fluoroscopic images employing fluorescent light emitted from fluorescing tissue, and X-ray transmission images. The corresponding live images may be obtained from imaging sensor 510, 574 when surgical site 550 is illuminated with suitable radiation from a visible light source; short wavelength visible or ultra-violet light source; and an X-ray source as illuminator respectively. Suitable short wavelength visible light may be, for example, one or more of blue light and violet light.

In FIG. 8, illuminator 576 and imaging sensor 574 are shown as housed together for the sake of convenience within in situ imager 570. In other embodiments, illuminator 576 and imaging sensor 574 may be housed separately and may be separately tagged with passive vectorized tracking markers of the same type as tracking markers 504, 507 and 572, and may be separately tracked by tracker 508. With illuminator 576 disposed in field of view 540 of tracker 508, system controller 520 may extract from the image information provided by tracker 508 the three-dimensional location and orientation of the tracking marker attached to illuminator 576 and display on monitor 530 an indication of where illuminator 576 will illuminate the patient at any given time. This allows the user to adjust the positioning of illuminator 576 proximate surgical site 550.

As with the embodiment of FIG. 5 and as described at the hand of FIG. 7, an additional instrument or implement 506, for example a hand piece that may be a dental drill, may be observed and tracked by tracker 508 of the monitoring system. To this end, implement 506 may bear a third passive vectorized tracking marker 507. As already explained at the hand of FIG. 6, the same arrangement may also be applied to non-dental surgery.

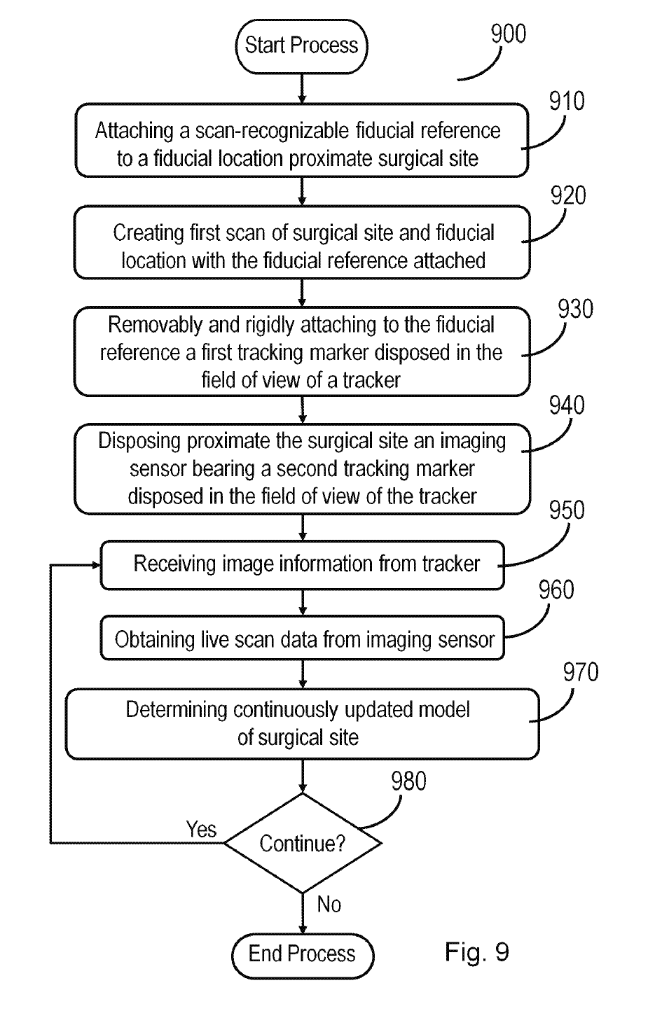

In another aspect, described at the hand of the flow chart of FIG. 9, a method [900] is provided for monitoring a surgical site 550, the method [900] comprising: removably attaching [910] passive vectorized fiducial reference 502 to a fiducial location proximate surgical site 550, the fiducial reference having a at least one of a marking and a shape perceptible on a scan; creating [920] prior to the surgical procedure a scan of surgical site 550 and the fiducial location with fiducial reference 502 attached; removably and rigidly attaching [930] to the fiducial reference 502 first passive vectorized tracking marker 504 disposed in field of view 540 of tracker 508; disposing [940] proximate surgical site 550 imaging sensor 510, 574 bearing second passive vectorized tracking marker 512, 572 disposed in the field of view of tracker 508; receiving [950] from tracker 508 image information of at least surgical site 550 and tracking markers 504, 512, 572; obtaining [960] from imaging sensor 510, 574 live images of surgical site 550; and determining [970] from the scan data, the image information, and the live images a continuously updated 3-dimensional model of surgical site 550 overlaid with live imagery of surgical site 550 as obtained by the imaging sensor.

After every image from imaging sensor 510, 574 has been overlaid on the scan data, the process may selectably return [980] to step [950] to receive new image information from tracker 508 and a corresponding new live image from imaging sensor 510, 574. The different kinds of imaging sensors 510, 574 and their modes of working have already been described above, as have illuminators 560, 576. Determining the continuously updated three-dimensional model of surgical site 550 comprises determining from the first scan data a three-dimensional location and orientation of vectorized fiducial reference 502 relative to the surgical site; and determining from the image information three-dimensional location and orientation information about first 504 and second 512, 572 passive vectorized tracking markers. In some embodiments, the determining the continuously updated three-dimensional model of surgical site 550 may further comprise determining from the image information three-dimensional location and orientation information about third passive vectorized tracking marker 507.

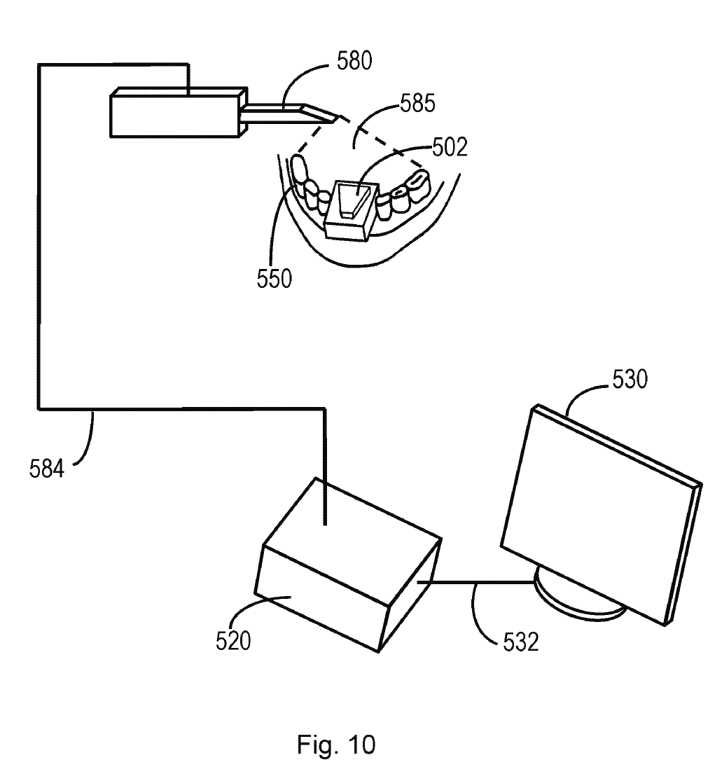

In another aspect of the invention, a monitoring system is described at the hand of FIG. 10, in which several elements present in FIG. 8 are employed. Instead of using in situ imager 570 of FIG. 8 that images only the surgical site 550, an intra-oral mapping device 580 is employed. Intra-oral mapping device 580 may be any device capable of obtaining intra-oral mapping information of a mapping region 585 covering simultaneously both surgical site 550 and single passive vectorized scan-visible fiducial 502. Intra-oral mapping device 580 may employ any suitable means, method, or radiation to obtain the intra-oral mapping information, including without limitation optical radiation, x-ray radiation, ultraviolet radiation, infrared radiation, or ultrasound radiation. The intra-oral mapping information may be a three dimensional. By way of example, intra-oral mapping device 580 may be any one of a number of different commercial devices generally referred to as “3D intra-oral scanners (IOS)”. Suitable IOS devices include, but are not limited to, the CS-3500 device supplied by Carestream of Atlanta, Ga.; the Lyhtos device supplied by Ormco of Orange, Calif.; and the MIA3D device supplied by DENSYS of Israel. These devices map the intra-oral region to obtain mapping information, usually employing optical radiation to do so, and then generate virtual three-dimensional intra-oral maps based on the mapping information. In general, intra-oral mapping device 580 may be any device capable of obtaining suitable mapping information to derive a virtual three-dimensional intra-oral image or map of suitable quality to allow the identification of the location and orientation of passive vectorized fiducial 502.

Miniaturization of these intra-oral mapping devices, together with the increases in computing capacity and speed, allow the development of devices of this type that are small enough to be disposed intra-orally while a procedure is undertaken using an implement 506. Implement 506 may be, for example, a dental drill. In some embodiments, implement 506 may bear a passive vectorized tracking marker 507 disposed to be visible to tracker 508. Intra-oral mapping device 580 is shown in FIG. 10 as a discrete device, but in some implementations miniaturization may allow intra-oral mapping device 580 to be integrated into implement 506.

In one implementation, described at the hand of FIG. 10, a prior scan of the surgical site 550 is performed in which fiducial reference 502 is rigidly disposed near the surgical site and is covered by the scan. As explained above, fiducial reference 502 may be rendered distinctly visible in the scan through higher imaging contrast by the employ of radio-opaque materials or high-density materials in the construction of fiducial reference 502. In other embodiments the material of the distinctive identifying and orienting markings may be created using suitable high density or radio-opaque inks or materials. This scan establishes the relative 3D spatial positions and orientations of surgical site 550 and fiducial reference 502. Intra-oral maps of mapping region 585 obtained by intra-oral mapping device 580 may be overlaid onto the pre-surgical scan data in order to determine the spatial relationship between the actual surgery and the interior of the surgical site, which may be invisible in the absence of a scan. A suitably miniaturized high computing speed intra-oral mapping device 580 may allow the spatial relationship between the actual surgery and the interior of the surgical site 550 to be tracked in real time during surgery. To this end, system controller 520 obtains intra-oral mapping information via mapping device data link 584 from intra-oral mapping device 580, and derives an intra-oral map from the intra-oral mapping information. In FIG. 10, mapping device data link 584 is shown as a wired link, but in other embodiments mapping device data link 524 may involve a radio, optical, or other suitable wireless link.

System controller 520 is programmable with software having instructions for configuring controller 520 for extracting from the intra-oral mapping information the 3D spatial location and orientation information of fiducial reference 502. The intra-oral map may alternatively or partially be derived from the intra-oral mapping information by intra-oral mapping device 580 using suitable internal processing. In either case, system controller 520 ultimately obtains an intra-oral map based on intra-oral mapping information obtained in turn by intra-oral mapping device 580. Controller 520, using suitable software with suitable instructions, may overlay the intra-oral map onto the pre-surgical scan data and may transmit the combined result via monitor link 532 to a display system, for example display monitor 530, on which it may be displayed. Fiducial 502 is the reference that may be used to orient all scans, mappings and imagery. Since there is no reference to any position or point outside the intra-oral zone, results displayed on monitor 530 are in this case not oriented with respect to any location outside the oral cavity of the patient. The operator adjusts the orientation of any imagery on monitor 530 to suit his or her needs. In this embodiment, high speed processing allows the updating of mappings and imagery in real time.

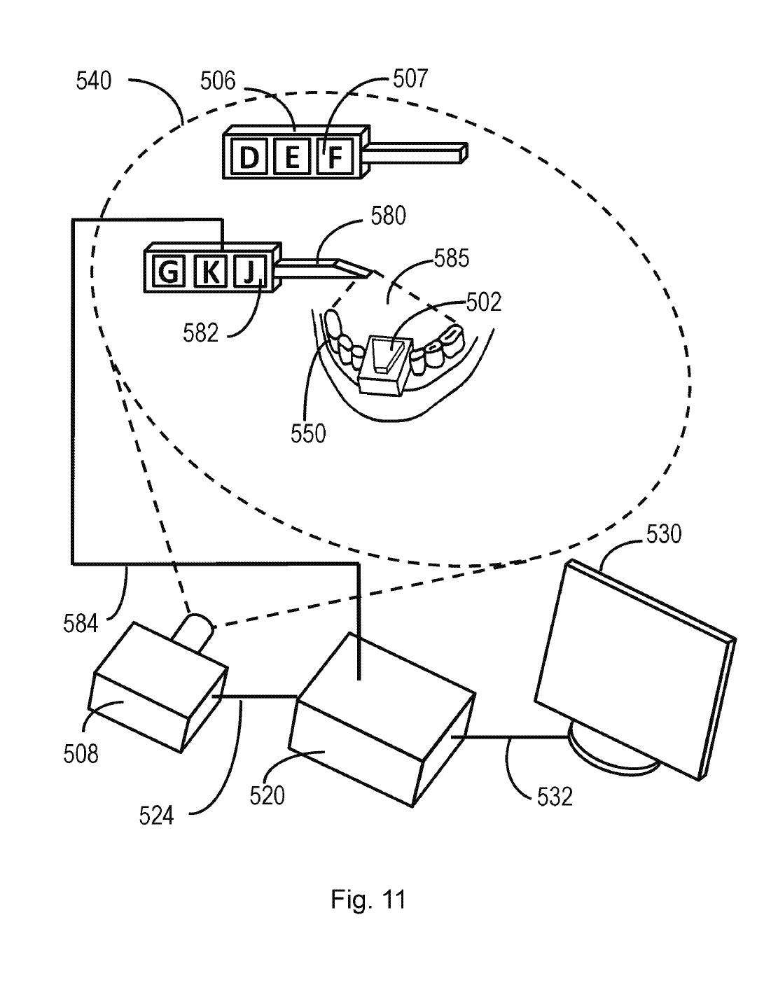

A first related embodiment, shown in FIG. 11, extends the system described at the hand of FIG. 10. In this extension of the embodiment, intra-oral mapping device 580 may bear a passive vectorized tracking marker 582 rigidly attached to intra-oral mapping device 580 in a known 3D position and orientation with respect to intra-oral mapping device 580. The known 3D position and orientation of marker 582 with respect to intra-oral mapping device 580 is stored in a memory of controller 520. In this embodiment, the system further comprises optical tracker 508 and tracking marker 582 is disposed in a field of view 540 of tracker 508 during the surgical procedure. Optical tracker 508 may be a non-stereo optical tracker. Tracker 508 communicates to controller 520 over tracker data link 524 image information of at least tracking marker 582. Controller 520, using suitable software instructions, extracts from the image information the current 3D position and orientation of marker 582. This allows controller 520 to orient in real space external to the patient the mutually superimposed imagery from the scan and from the mapping, and to display the result on monitor 530. With suitably high speed processing, this information may be updated in real time on monitor 530.

The system may further comprise a surgical implement 506 disposed in field of view 540 of tracker 508 and having a working tip Implement 506 may bear a rigidly attached passive vectorized tracking marker 507 disposed in field of view 540 of non-stereo optical tracker 508. The location and orientation of the working tip of implement 506 relative to passive vectorized tracking marker 507 on implement 506 is known and is stored in a memory of controller 520 before surgery. This allows controller 520 to determine the location and orientation of a working tip of implement 506 and to display it on monitor 530 along with the joint imagery from the scan and the mapping described above. The use of high speed processing may allow working tip of implement 506 to be tracked in real time and displayed in real time with the joint imagery on monitor 530. This embodiment facilitates the real time display of the joint imagery of the scan and the mapping on monitor 530 during a surgical procedure without employing tracking markers attached to a patient. Only the fiducial reference 502 has to be attached to the patient for tracking. It allows both the intra-oral mapping device 580 and the implement 506 to be tracked in real external space while displaying the joint imagery and the progress of surgery.

In an alternative arrangement to that of FIG. 11, instead of a separate intra-oral mapping device 580 and a separate surgical device 506, a surgical implement having a working tip may be integrated into the intra-oral mapping device 580 so that passive vectorized tracking marker 582 has a fixed three-dimensional spatial relationship with the working tip of the surgical implement. The software program may comprise a further series of instructions which when executed by the processor, for example processor 214 of FIG. 2, determines from the real time information of tracking marker 582 a current position and orientation of marker 582 and relates a position and orientation of the working tip of the surgical implement to the surgical site 550 based on the real time information of tracking marker 582.

To this end, the relative position and orientation of tracking marker 582 with respect to both device 580 and the working tip of implement 506 may be stored in a memory associated with controller 520. In this embodiment tracker 508 need only track marker 582 in three dimensions in order to obtain not only the position and orientation of the working tip of implement 506, but also that of fiducial 502. This allows the pre-surgical scan data and any intra-oral map obtained by controller 520 via device 580 to be mapped onto each other and displayed in real time on monitor 530, correctly positioned with respect to the working tip of implement 506 and oriented in respect of the real space external to the patient. In this arrangement, tracking marker 507 may be redundant, as only one tracking marker is required on the integrated device.

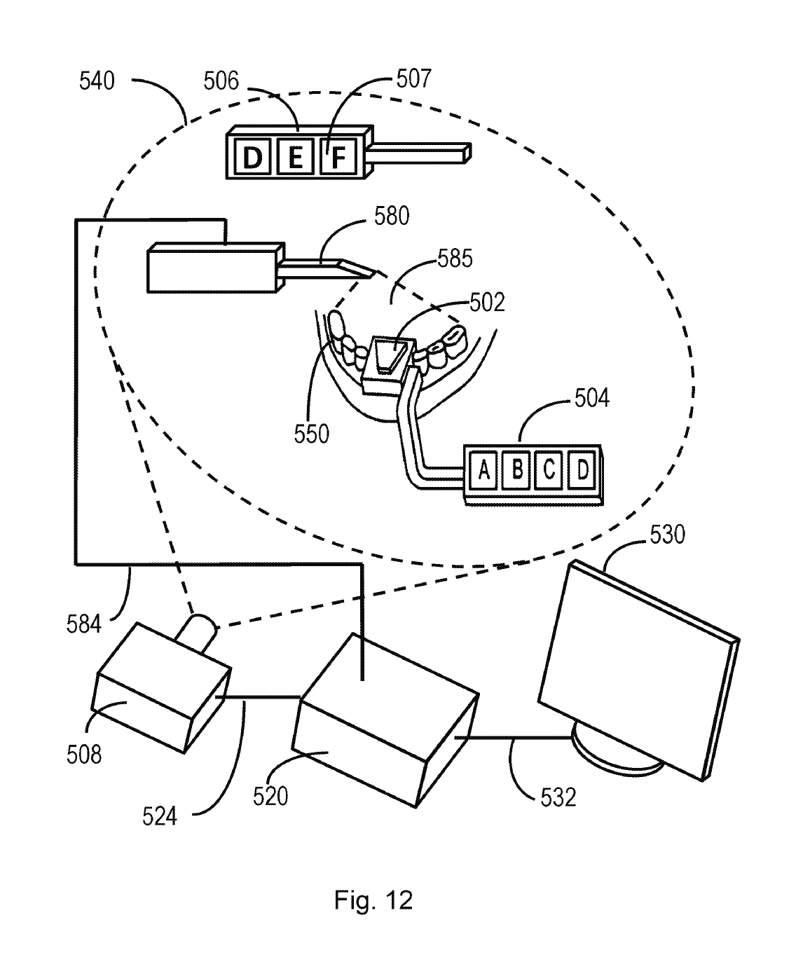

A second related embodiment, shown in FIG. 12, extends the system of FIG. 10. This embodiment employs passive vectorized tracking marker 504 disposed rigidly with respect to fiducial reference 502 via a suitable tracking pole. Tracking marker 504 is disposed within field of view 540 of non-stereo optical tracker 508. The use of such tracking poles has been described above at the hand of FIGS. 3A-J. The 3D spatial orientation and position of fiducial 502 relative to tracking marker 504 is stored in a memory of controller 520. Therefore, knowledge of the 3D spatial position and orientation of tracking marker 504 directly allows controller 520 to determine the 3D spatial location and orientation of fiducial 502 relative to non-stereo optical tracker 508. The mapping information obtained by intra-oral mapping device 580 covers both surgical site 550 and single passive vectorized fiducial 502 and therefore allows any map derived from the mapping information to be oriented and located relative to fiducial 502, and thereby relative to tracking marker 504. The intra-oral map may therefore be spatially mapped in three dimensions onto the pre-surgical scan data and the joint imagery so obtained may be displayed on monitor 530 in spatial reference to non-stereo optical tracker 508. As with previous embodiments, the use of high speed processing may allow this joint imagery to be updated in real time on monitor 530.

The system of FIG. 12 further comprises surgical implement 506, as described above, having a working tip Implement 506 may, as above, bear a rigidly attached passive vectorized tracking marker 507 disposed in field of view 540 of non-stereo optical tracker 508. The location and orientation of a working tip of implement 506 relative to passive vectorized tracking marker 507 on implement 506 is known and is stored in a memory of controller 520. This allows controller 520 to determine the location and orientation of a working tip of implement 506 and to display it on monitor 530 along with the joint imagery from the scan and the mapping described above. As with previous embodiments, the use of high speed processing may allow working tip of implement 506 to be tracked in real time and displayed in real time with the joint imagery on monitor 530.

In an alternative to the arrangement of FIG. 12, implement 506 physically comprises intra-oral mapping device 580 in mutually fixed position and orientation so that knowledge of the three dimensional location and orientation of passive vectorized tracking marker 507 on implement 506 implies that the location and orientation of device 580 is thereby also known. To this end, the relative position and orientation of tracking marker 507 with respect to both device 580 and the working tip of implement 506 may be stored in a memory associated with controller 520. In this embodiment tracker 508 need only track marker 507 in three dimensions in order to obtain not only the position and orientation of the working tip of implement 506, but also that of fiducial 502. This allows the pre-surgical scan data and any intra-oral map obtained by controller 520 via device 580 to be mapped onto each other and displayed in real time on monitor 530, correctly positioned with respect to the working tip of implement 506 and oriented in respect of the real space external to the patient.

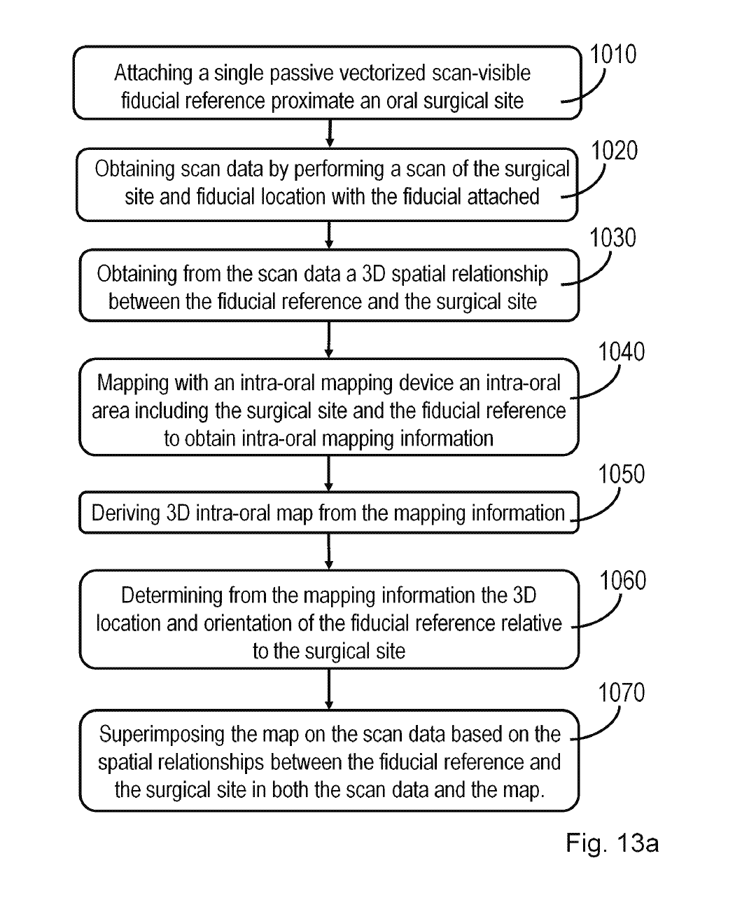

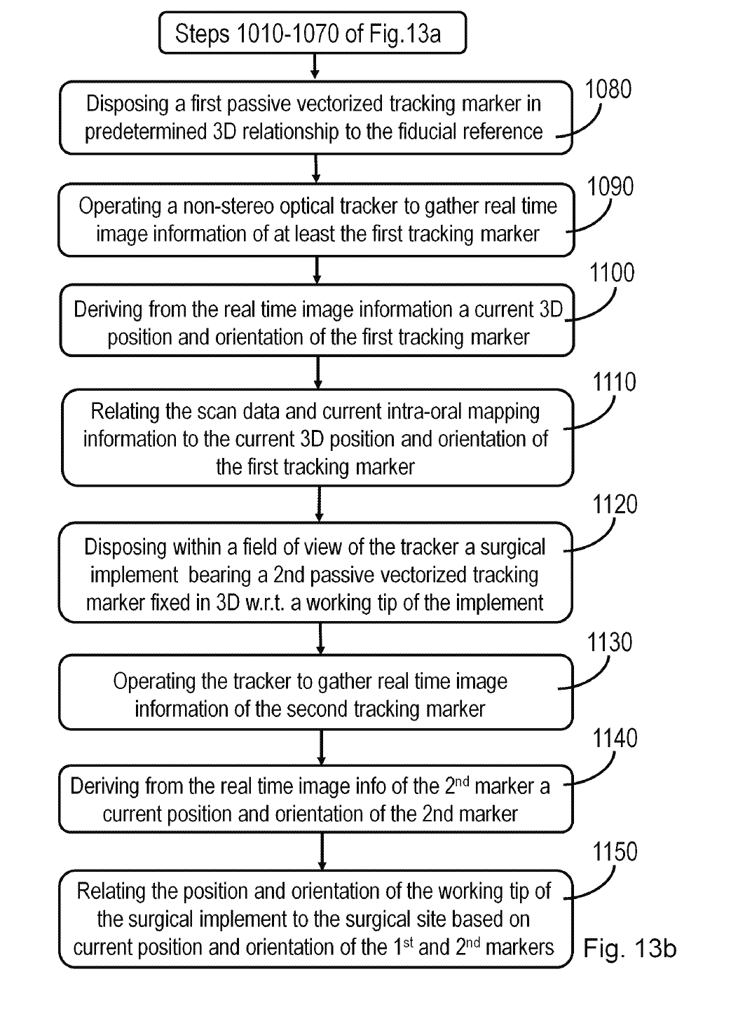

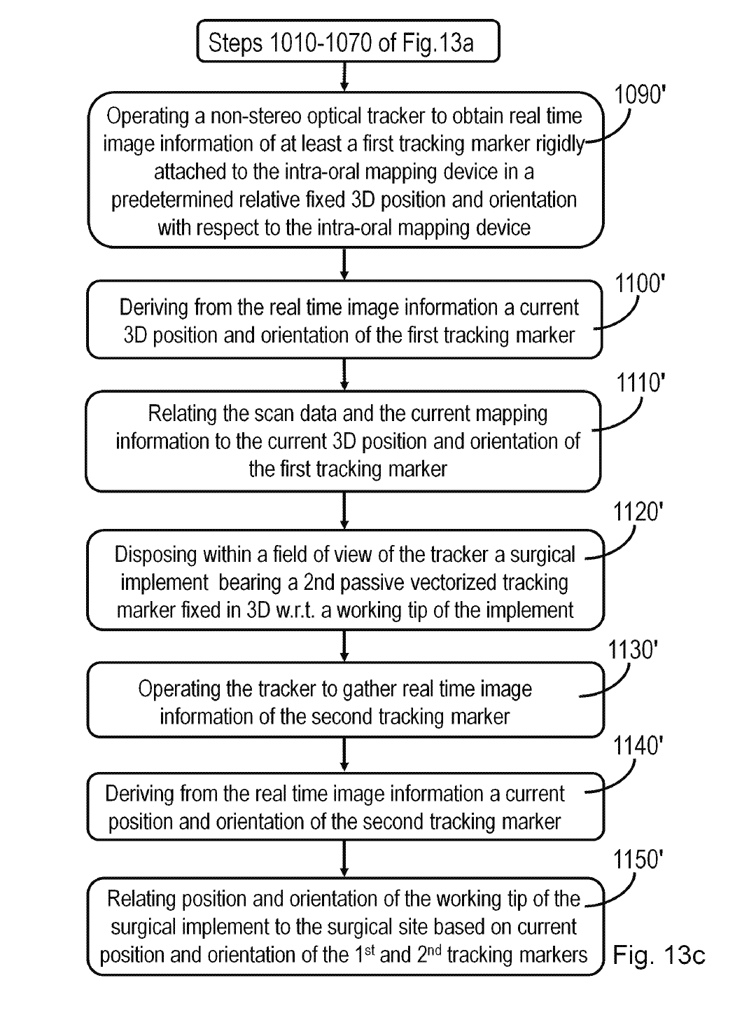

In a further aspect, a method is provided for superimposing three dimensional intra-oral mapping information on pre-surgical scan data, as described at the hand of FIGS. 10, 11, 12, 13a, 13b and 13c. The method comprises (see FIGS. 13a and 10): removably and rigidly attaching [1010] a single passive vectorized scan-visible fiducial reference 502 proximate an oral surgical site 550 of a surgical patient; performing a pre-surgical scan of the surgical site 550 with the fiducial reference 502 attached to obtain [1020] the scan data; obtaining [1030] from the scan data the three-dimensional spatial relationship between the fiducial reference 502 and the surgical site 550; mapping [1040] by means of an intra-oral mapping device 580 an intra-oral area 585 of the patient including the surgical site 550 and the fiducial reference 502 to obtain mapping information about the surgical site 550 and the fiducial reference 502; deriving [1050] from the mapping information a three-dimensional intra-oral map of the intra-oral area 585; determining [1060] from the mapping information the spatial location and orientation of the fiducial reference 502 relative to the surgical site 550; and superimposing [1070] the intra-oral map on the pre-surgical scan data based on the spatial relationship between the fiducial reference 502 and the surgical site 550 in the scan data and the spatial relationship between the fiducial reference 502 and the surgical site 550 in the intra-oral map. The method may further comprise displaying the superimposed intra-oral map and the pre-surgical scan data on a display system 530. The mapping, deriving, determining, superimposing and displaying may be done in real time.

In a first extension of the method provided here (see FIGS. 13b and 12), the method may further comprise rigidly and removably disposing [1080] a first passive vectorized tracking marker 504 in a predetermined fixed three-dimensional spatial position and orientation relative to the single fiducial reference 502; operating [1090] a non-stereo optical tracker 508 to gather real time image information of at least the first tracking marker 504; deriving [1100] from the real time image information of the first tracking marker 504 a current three-dimensional spatial position and orientation of the first tracking marker 504; and relating [1110] the scan data and current intra-oral mapping information to the current three-dimensional spatial position and orientation of the first tracking marker 504.

The same first extension of the method may further comprise disposing [1120] within a field of view 540 of the tracker 508 a surgical implement 506 bearing a second passive vectorized tracking marker 507 in fixed three-dimensional spatial relationship with a working tip of the surgical implement 506; operating [1130] the tracker 508 to gather real time image information of the second tracking marker 507; deriving [1140] from the real time image information of the second tracking marker 507 a current position and orientation of the second tracking marker 507; and relating [1150] a position and orientation of a working tip of the surgical implement 506 to the surgical site 550 based on the real time information of the first 504 and second 507 tracking markers. The disposing a surgical instrument may comprise disposing surgical instrument 506 wherein the intra-oral mapping device 580 is integrated into surgical instrument 506, the intra-oral mapping device 580 having a known fixed spatial relationship with the second passive vectorized tracking marker 507.

In a second extension of the method provided here (see FIGS. 13c and 11) the method may further comprise operating [1090′] a non-stereo optical tracker 508 to obtain real time image information of at least a first tracking marker 582 rigidly attached to the mapping device 580 in a predetermined relative fixed three-dimensional spatial position and orientation with respect to the mapping device 580; deriving [1100′] from the real time image information of the first tracking marker 582 a current three-dimensional spatial position and orientation of the first tracking marker 582; relating [1110′] the scan data and the current intra-oral mapping information to the current three-dimensional spatial position and orientation of the first tracking marker 582.

The same second extension of the method may comprise disposing [1120′] within a field of view 540 of the tracker 508 a surgical implement 506 bearing a second passive vectorized tracking marker 507 in fixed three-dimensional spatial relationship with a working tip of the surgical implement 506; operating [1130′] the non-stereo optical tracker 508 to gather real time image information of the second tracking marker 507; deriving [1140′] from the real time image information of the second tracking marker 507 a current position and orientation of the second tracking marker 507; and relating [1150′] a position and orientation of the working tip of the surgical implement 506 to the surgical site 550 based on the real time information of the second tracking marker 507.

The same second extension may instead comprise disposing within a field of view 540 of the tracker 508 a surgical implement integrated with the intra-oral mapping device 580, the first tracking marker 582 having a known and fixed spatial relationship with a working tip of the surgical implement; and relating a position and orientation of the working tip of the surgical implement to the surgical site 550 based on the real time information of the first tracking marker 582.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A monitoring system for a surgical site comprising:
a single passive vectorized scan-visible fiducial reference adapted to be fixed proximate an oral surgical site of a surgical patient;
an intra-oral mapping device adapted to be disposed intra-orally proximate the surgical site and adapted to obtain current mapping information of an intra-oral mapping area including the surgical site and the fiducial reference;
pre-surgical scan data of the surgical site with the fiducial reference fixed proximate the surgical site, the scan data including the fiducial reference;
a controller in data communication with the intra-oral mapping device and comprising a processor with memory and a software program comprising a plurality of instructions which when executed by the processor determines from the current mapping information a current three-dimensional spatial position and orientation of the fiducial reference relative to the intra-oral mapping device, and spatially relates the scan data to the current mapping information based on the current three-dimensional spatial position and orientation of the single fiducial reference; and
a display system in data communication with the controller and adapted to display the current mapping information of the surgical site superimposed on the scan data.

2. The monitoring system of claim 1, further comprising:
a first passive vectorized tracking marker rigidly and removably disposed in a predetermined fixed three-dimensional spatial position and orientation relative to the single fiducial reference;
a non-stereo optical tracker in data communication with the controller and configured and disposed for obtaining from a field of view of the tracker real time image information of at least the first tracking marker; and
the software program comprising a further series of instructions which when executed by the processor determines from the real time image information a current three-dimensional spatial position and orientation of the first tracking marker, and relates the scan data and current intra-oral mapping information to the current three-dimensional spatial position and orientation of the first tracking marker.

3. The monitoring system of claim 2, further comprising a surgical implement bearing a second passive vectorized tracking marker in fixed three-dimensional spatial relationship with a working tip of the surgical implement and disposed within the field of view of the tracker, wherein
the real time image information of at least the first tracking marker further includes information of the second tracking marker, and
the software program comprises yet a further plurality of instructions which when executed by the processor determines from the real time image information current positions and orientations of the second tracking marker, and relates a position and orientation of a working tip of the surgical implement to the surgical site based on the real time information of the second tracking marker.

4. The monitoring system of claim 3, wherein the intra-oral mapping device is integrated into the surgical implement and the second passive vectorized tracking marker has a fixed three-dimensional spatial relationship with the intra-oral mapping device.

5. The monitoring system of claim 1, further comprising:
a first passive vectorized tracking marker rigidly attached to the intra-oral mapping device in a predetermined relative fixed three-dimensional spatial position and orientation with respect to intra-oral mapping device; and
a non-stereo optical tracker in data communication with the controller and configured and disposed for obtaining from a field of view of the tracker real time image information of at least the first tracking marker;
the software program comprising a second series of instructions which when executed by the processor determines from the real time image information of the first tracking marker a current three-dimensional spatial position and orientation of the first tracking marker, and relates the scan data and the current intra-oral mapping information to the current three-dimensional spatial position and orientation of the first tracking marker.

6. The monitoring system of claim 5, further comprising a surgical implement bearing a second passive vectorized tracking marker in fixed three-dimensional spatial relationship with a working tip of the surgical implement and disposed within the field of view of the tracker, wherein
the real time image information of at least the first tracking marker further includes information of the second tracking marker, and
the software program comprises a plurality of instructions which when executed by the processor determines from the real time image information a current position and orientation of the second tracking marker, and relates a position and orientation of a working tip of the surgical implement to the surgical site based on the real time information of the second tracking marker.

7. The monitoring system of claim 5, further comprising a surgical implement having a working tip, and wherein
the surgical implement is integrated into the intra-oral mapping device;
the first passive vectorized tracking marker has a fixed three-dimensional spatial relationship with the working tip of the surgical implement; and
the software program comprises a third series of instructions which when executed by the processor determines from the real time information of the first tracking marker a current position and orientation of the first tracking marker and relates a position and orientation of the working tip of the surgical implement to the surgical site based on the real time information of the first tracking marker.

8. A method for superimposing three dimensional intra-oral mapping information on pre-surgical scan data, the method comprising:
removably and rigidly attaching a single passive vectorized scan-visible fiducial reference proximate an oral surgical site of a surgical patient;
performing a pre-surgical scan of the surgical site with the fiducial reference attached to obtain the scan data;
obtaining from the scan data the three-dimensional spatial relationship between the fiducial reference and the surgical site;
mapping by means of an intra-oral mapping device an intra-oral area of the patient including the surgical site and the fiducial reference to obtain mapping information about the surgical site and the fiducial reference;
deriving from the mapping information a three-dimensional intra-oral map of the intra-oral area;
determining from the mapping information the spatial location and orientation of the fiducial reference relative to the surgical site; and
superimposing the intra-oral map on the pre-surgical scan data based on the spatial relationship between the fiducial reference and the surgical site in the scan data and the spatial relationship between the fiducial reference and the surgical site in the intra-oral map.

9. The method of claim 8, further comprising displaying the superimposed intra-oral map and the pre-surgical scan data on a display system.

10. The method of claim 9, wherein the mapping, deriving, determining, superimposing and displaying are done in real time.

11. The method of claim 8, further comprising:
rigidly and removably disposing a first passive vectorized tracking marker in a predetermined fixed three-dimensional spatial position and orientation relative to the single fiducial reference;
operating a non-stereo optical tracker to gather real time image information of at least the first tracking marker;
deriving from the real time image information of the first tracking marker a current three-dimensional spatial position and orientation of the first tracking marker; and
relating the scan data and current intra-oral mapping information to the current three-dimensional spatial position and orientation of the first tracking marker.

12. The method of claim 11, further comprising:
disposing within a field of view of the tracker a surgical implement bearing a second passive vectorized tracking marker in fixed three-dimensional spatial relationship with a working tip of the surgical implement;
operating the tracker to gather real time image information of the second tracking marker;
deriving from the real time image information a current position and orientation of the second tracking marker; and
relating a position and orientation of a working tip of the surgical implement to the surgical site based on the real time information of the first and second tracking markers.

13. The method of claim 12, wherein the disposing a surgical instrument comprises disposing surgical instrument wherein the intra-oral mapping device is integrated into surgical instrument, the intra-oral mapping device having a known fixed spatial relationship with the second passive vectorized tracking marker.

14. The method of claim 8, further comprising:
operating a non-stereo optical tracker to obtain real time image information of at least a first tracking marker rigidly attached to the mapping device in a predetermined relative fixed three-dimensional spatial position and orientation with respect to the mapping device;
deriving from the real time image information of the first tracking marker a current three-dimensional spatial position and orientation of the first tracking marker;
relating the scan data and the current intra-oral mapping information to the current three-dimensional spatial position and orientation of the first tracking marker.

15. The method of claim 14, further comprising:
disposing within a field of view of the tracker a surgical implement bearing a second passive vectorized tracking marker in fixed three-dimensional spatial relationship with a working tip of the surgical implement;
operating the non-stereo optical tracker to gather real time image information of the second tracking marker;
deriving from the real time image information of the second tracking marker a current position and orientation of the second tracking marker; and
relating a position and orientation of the working tip of the surgical implement to the surgical site based on the real time information of the second tracking marker.

16. The method of claim 14, further comprising:
disposing within a field of view of the tracker a surgical implement integrated with the intra-oral mapping device, the first tracking marker having a known and fixed spatial relationship with a working tip of the surgical implement; and
relating a position and orientation of the working tip of the surgical implement to the surgical site based on the real time information of the first tracking marker.
